(19) United States
(12) Patent Application Publication
(10) Pub. No.: US 2020/0234827 A1
(43) Pub. Date: Jul. 23, 2020

(54) METHODS AND SYSTEMS FOR DIAGNOSING AND TREATING DISORDERS

(71) Applicant: Mira Therapeutics, Inc., Wilmington, DE (US)

(72) Inventors: Charles J. Internicola, Matawan, NJ (US); Gregory Mercado, Hoboken, NJ (US); Annika H. Roll, Brewster, NY (US); Seth J. Kirschner, Long Island City, NY (US); Nicholas Gattuso, Toms River, NJ (US)

(21) Appl. No.: 16/749,812

(22) Filed: Jan. 22, 2020

Related U.S. Application Data
(60) Provisional application No. 62/795,271, filed on Jan. 22, 2019.

(51) Int. Cl. G16H 50/20; G16H 50/50; G16H 70/20; G16H 20/70; A61B 5/16; G06F 3/01; G06F 3/0482

(52) U.S. Cl. CPC G16H 50/20; G16H 50/50; G16H 70/20; G06F 3/0482; A61B 5/165; G06F 3/011; G16H 20/70

(57) ABSTRACT

Provided herein are methods and systems for diagnosing, managing, and treating disorders and conditions related to psychological trauma, such as Post-Traumatic Stress Disorder (PTSD), and other mental health conditions with similar symptoms through self-management tools, user insights, and personalized therapies. A system consisting of a User interface, a Data Storage and Processing Architecture and a Therapist Interface for self-management and treatment support of mental health conditions such as PTSD or other conditions with similar symptoms may be provided to a user via an electronic device, such as a mobile device, computing device, tablet, or the like.

### CROSS-REFERENCE TO RELATED APPLICATION

This application claims priority to U.S. Provisional Application No. 62/795,271, filed Jan. 22, 2019, which is incorporated by reference herein in its entirety.

### BACKGROUND

People suffering from Post-Traumatic Stress Disorder, or from similar mental or psychiatric disorders or conditions, often experience a range of symptoms such as: flashbacks, anxiety, panic attacks, hyperarousal, and dissociation. According to Mental Health America, dissociation is defined as “a lack of connection in a person's thoughts, memory, and sense of identity.” To manage these symptoms, sufferers can use “grounding techniques” that prevent, dull, or distract from these symptoms. When experiencing the symptoms, sufferers can consciously use grounding techniques on themselves, or ask another individuals to guide them through such grounding techniques. Therapists teach their clients functions and techniques, including grounding techniques, to manage crises. However, a problem arises when a therapist is not immediately present to guide a client through identifying particular symptoms and thought patterns or other diagnostic or therapeutic functions, The use of grounding techniques is dependent on: a) the person knowing and remembering the techniques, and b) the person having enough control and clarity of mind to use them when the symptoms arise. Mental control and clarity of mind are impaired by the nature of the symptoms themselves. Symptoms can occur at any time, often due to unknown reasons, and often when the person is alone or without someone who understands the person's situation and can help. Therefore, sufferers have a need to self-manage through these symptoms. These symptoms can lead to undesirable outcomes such as anxiety attacks, amnesia, drug addiction, and suicidal thoughts.

Mental health professionals utilize various forms of psychotherapy to treat PTSD and other similar mental disorders. According to Psychcentral.com, Psychotherapy (also called “therapy”) “is a process whereby psychological problems are treated through communication and relationship factors between an individual and a trained mental health professional.” Psychotherapy aims at helping a patient eliminate or control the symptoms of mental illnesses and emotional problems. To deliver effective therapy, therapists must first identify particular symptoms and thought patterns that their client experiences. Therapists identify such information by interviewing their clients during sessions, and by asking their clients to journal their symptoms on a daily basis.

One of the treatment goals for PTSD and similar mental conditions is that sufferers develop normal reactions to their “triggers.” According to WebMD, “triggers are sights, sounds, smells, or thoughts that remind a person of a traumatic event.” Triggers can initiate or escalate symptoms like dissociation, anxiety, and depression, among others. Although a person may have a large number of triggers, she may not be able to identify which specific stimuli triggers them. Because so many people struggle to identify all, or even some, of their triggers, most sufferers cannot adequately discuss their triggers with their therapist. Thereby impacting negatively the potential for effective treatment choices and faster recovery.

Measurement-based care (MBC) is the practice of continually gathering data and information about a patient's symptoms for the purpose of diagnostic and therapeutic care, and modifying treatment according to the results. MBC relies on symptom and wellness assessments taken either before, during, or after treatment sessions. By objectively evaluating a patient's progress, therapists can continue or modify treatment methods based on that patient's aggregate assessment data. While utilizing MBC in a therapeutic setting has been proven to substantially improve therapy outcomes, the current technology available makes it difficult for clinicians to successfully implement MBC. Some problems identified by clinicians in implementing MBC include time restrictions during therapy sessions, limited technological, organizational, and financial resources, complexity in patients' experiences and diagnoses, and procedural training required to implement MBC.

These and other shortcomings are addressed by the present disclosure.

### SUMMARY

Methods, systems, and apparatuses are described for diagnosing and treating, among other things, mental disorders by way of various diagnostic tools and principles as well as therapeutic tools and principles by way of data-gathering, diagnosis, and treatment of trauma-related mental disorders such as PTSD as well as other disorders.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of the present description serve to explain the principles of the methods and systems described herein:

FIG. 1 illustrates an example computing environment in which the disclosed methods and systems may be executed;

FIG. 2 illustrates an example computing device;

FIGS. 3A-3E illustrate exemplary configurations and wireframes for carrying out the described methods;

FIG. 4 illustrates an example system;

FIG. 5 illustrates an interface hierarchy;

FIG. 6 illustrates an example communication session;

FIGS. 7A-7CC illustrate example interfaces;

FIG. 8 illustrates an example process flow;

FIGS. 9A-9F illustrate exemplary process flows;

FIG. 10 illustrates a flowchart of an example method; and

FIG. 11 illustrates an example computing environment in which the disclosed methods and systems may be executed.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Hereinafter, various embodiments of the present disclosure will be described with reference to the accompanying drawings. As used herein, the term “user” may indicate a person who uses an electronic device or a device (e.g., an artificial intelligence electronic device) that uses an electronic device.

Virtual Reality (VR) may refer to three-dimensional (3D) rendered, interactive videos. Such videos (which may also be called “experiences”) are viewed using wearable VR Headsets. The VR environment may force users to focus on the visual space surrounding them, restricting their sensory input. This is an ideal environment for administering exposure therapy because it limits the distractions that can occur. While several VR exposure therapy tools exist, they have limitations including special and temporal limitations, an inability to customize treatment on an individual level, and other limitations.

Augmented Reality (AR) may refer to hybrid visualizations comprising elements of a physical world as well as elements of a virtual world. AR experiences may incorporate stimuli from the physical world as well as additional stimuli generated by, for example, a computing device. Such AR elements may be complementary to the physical world stimuli.

Described herein are methods, systems, and apparatus for management of symptoms related to PTSD and other mental health disorders; tracking of these symptoms and of the health condition; identifying and analyzing triggers, progress, and treatments; supporting treatment decisions; and allowing therapists to deliver personalized and customized therapy, such as VR Exposure Therapy, to their clients. The systematic exposure to triggers is a broadly accepted treatment option to treat a variety of mental disorders, including PTSD. The concept of exposure is at the core of many psychotherapy models, including Prolonged Exposure Therapy, eye movement desensitization and reprocessing (EMDR) therapy, Narrative Exposure Therapy, and other Cognitive Behavioral therapies. Exposure therapy primarily breaks down into two types: imaginal exposure and in vivo exposure. In vivo exposure may involve directly facing a feared object, situation or activity in real life, and imaginal exposure may involve vividly imagining the feared object, situation, or activity. As part of this treatment model, therapists may utilize a variety of diagnostic and therapeutic functions including but not limited to writing exercises, pictures, videos, real-life experiences, and augmented reality (AR) or virtual reality (VR) experiences to safely provide exposure. The goal of this treatment is to ameliorate the symptoms of PTSD by helping sufferers develop a normal reaction to their triggers. The use of exposure therapy and its effectiveness however, may be limited by the lack of fidelity in the reproduction of triggers, and by the lack of identification of the actual trigger. The present disclosure addresses these shortcomings and others.

For example, using the methods and systems described herein, a user may daily record his or her symptoms and may further rate the severity of the symptoms. Additionally, a user may journal experiences, such as an experience which caused a flashback or other symptom. It is not uncommon for daily circumstances to cause a trauma-survivor to revisit a traumatic experience. For example, a combat veteran may hear a car backfire, or a jackhammer at a construction site, and the noise may cause the combat veteran to flashback to a combat scene. When a user uses the journal function, certain data may be recorded automatically, for instance temporal information such as time and date, as well as spatial information such as location. Additionally, user identifiers may be recorded so as to provide a custom therapy exercise or experience. By recording and reflecting on their experiences, clients can recall information more accurately and communicate more effectively during therapy sessions. This facilitates the therapeutic function of reprocessing, or reforming a reaction to an experience and thereby changing the way a user may experience any given set of circumstance or recollections. In the present methods and systems, reprocessing may be guided by a therapist or may be guided by artificial intelligence (AI). Further, reprocessing and other diagnostic and therapeutic functions may be guided by a digital assistant. Many therapists consider journaling to be an indispensable part of recovery. However, many sufferers do not journal or do so very sporadically. Journaling may provide powerful insight for treatment of individuals as well as populations suffering traumatic disorders. Among other reasons, sufferers do not journal their symptoms and experiences because: the process is inconvenient, they do not have access to their journal or recording device, they do not know which details to record, and their memory is inhibited by the symptoms themselves. The present methods, systems, and apparatus facilitate journaling by allowing users to record experiences when it is convenient for the user.

In addition to journaling, the present methods may allow a user to execute therapeutic functions such as grounding exercises, experiential exercises or AR/VR experiences. Grounding exercises may comprise color-finding or color-matching exercises, breathing exercises, haptic or tactile exercises, and the like. Experiential exercises may comprise revisiting, re-experiencing, or re-processing traumatic events or triggering events such as events which caused a flashback. Further the methods and systems described herein may provide therapeutic experiences such as calming visuals or sounds.

Accordingly, several advantages of the present methods and systems are as follows:

To provide management tools that are accessible whenever symptoms occur, that can be used both when the user is alone or with other people, that can be activated and completed also in cases where the user's cognitive ability and self-control may be somewhat reduced or impaired;

To log information on the use of management tools in the system, improving symptom tracking and reducing the reliance on the user's memory;

To provide convenient journaling and reporting tools to help users and therapists identify triggers and progress;

To provide insights from the analyses on collected data to inform on treatment effect, inform treatment decisions, and create personalized therapies;

To support the delivery of diagnostic or therapeutic measurement-based care;

Providing users with self-management tools that are accessible when experiencing symptoms anytime, anywhere;

Activating and completing self-management/grounding exercises can be done without full cognitive control by the user;

Reducing the reliance on the user's memory for symptom tracking, identification of triggers, and identification of effective treatments and techniques;

Providing unique insights to inform treatment decisions and treatment optimization, supporting the use of measurement-based care, that has been proven the most effective in the treatment of mental health disorders;

Providing unique information for personalization of therapies, such as AR/VR exposure therapies (e.g., experiences); and

Reducing the limitations and challenges on self-management of symptoms and improving information for treatment decisions and personalization, which may all users to benefit from less severe symptoms and faster recovery.

While the description may contain many specifics, these should not be construed as limitations on the scope, but rather as an exemplification of several embodiments thereof. Many other variations are possible. For example the grounding exercises may have additional exercises or new exercises may replace existing ones, the user interface may add new information sources, and offer communities and forums to connect to other users. Other ramifications may add connections to established services, such as teletherapy.

Users may be individuals suffering from Post-Traumatic Stress Disorder or from another mental health condition with similar symptoms. Such other conditions may include: obsessive-compulsive disorder, phobias, dissociative amnesia, depersonalization-derealization disorder, dissociative identity disorder, etc. The user interface helps users self-manage and track symptoms such as avoidance symptoms, dissociation symptoms, mood changes, hypervigilance symptoms, flashbacks, dysphoria, nightmares, anxiety, depression, hyperarousal, avoidance, depersonalization, derealization, anger, amnesia, identity confusion, irritability, insomnia, substance abuse, self-harm, phantom pains, physical pains, headaches, etc.

Since the user interface is readily available to the user anytime and anywhere, it can be used when symptoms occur. And because the User interface is easily activated, and interactive, it can be used even when the user's cognitive abilities may be somewhat impaired or reduced.

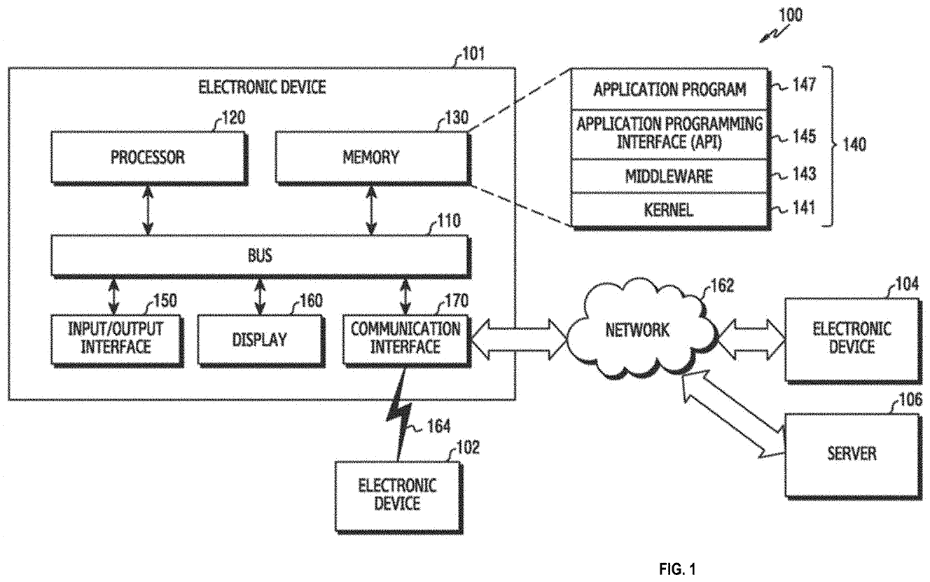

FIG. 1 illustrates a network environment including an electronic device configured for diagnosing and treating mental disorders such as PTSD. Referring to FIG. 1, an electronic device 101 in a network environment 100 is disclosed according to various exemplary embodiments. The electronic device 101 may include a bus 110, a processor 120, a memory 130, an input/output interface 150, a display 160, and a communication interface 170. In a certain exemplary embodiment, the electronic device 101 may omit at least one of the aforementioned constituent elements or may additionally include other constituent elements. The electronic device 101 may be, for example, a mobile phone, a tablet computer, a laptop, a desktop computer, a smartwatch, and the like.

The bus 110 may include a circuit for connecting the aforementioned constituent elements 110 to 170 to each other and for delivering communication (e.g., a control message and/or data) between the aforementioned constituent elements.

The processor 120 may include one or more of a Central Processing Unit (CPU), an Application Processor (AP), and a Communication Processor (CP). The processor 120 may control, for example, at least one of other constituent elements of the electronic device 101 and/or may execute an arithmetic operation or data processing for communication. The processing (or controlling) operation of the processor 120 according to various embodiments is described in detail with reference to the following drawings.

The memory 130 may include a volatile and/or non-volatile memory. The memory 130 may store, for example, a command or data related to at least one different constituent element of the electronic device 101. According to various exemplary embodiments, the memory 130 may store a software and/or a program 140. The program 140 may include, for example, a kernel 141, a middleware 143, an Application Programming Interface (API) 145, and/or an application program (or an “application”) 147, or the like. The application program 147 may be a diagnostic or therapeutic program or combination thereof, configured for controlling one or more functions of the electronic device 101 and/or an external device. At least one part of the kernel 141, middleware 143, or API 145 may be referred to as an Operating System (OS). The memory 130 may include a computer-readable recording medium having a program recorded therein to perform the method according to various embodiment by the processor 120.

The kernel 141 may control or manage, for example, system resources (e.g., the bus 110, the processor 120, the memory 130, etc.) used to execute an operation or function implemented in other programs (e.g., the middleware 143, the API 145, or the application program 147). Further, the kernel 141 may provide an interface capable of controlling or managing the system resources by accessing individual constituent elements of the electronic device 101 in the middleware 143, the API 145, or the application program 147.

The middleware 143 may perform, for example, a mediation role so that the API 145 or the application program 147 can communicate with the kernel 141 to exchange data.

Further, the middleware 143 may handle one or more task requests received from the application program 147 according to a priority. For example, the middleware 143 may assign a priority of using the system resources (e.g., the bus 110, the processor 120, or the memory 130) of the electronic device 101 to at least one of the application programs 147. For instance, the middleware 143 may process the one or more task requests according to the priority assigned to the at least one of the application programs, and thus may perform scheduling or load balancing on the one or more task requests.

The API 145 may include at least one interface or function (e.g., instruction), for example, for file control, window control, video processing, or character control, as an interface capable of controlling a function provided by the application 147 in the kernel 141 or the middleware 143.

The API may gather, store, encrypt, package, send, and/or authenticate patient report files. The API may securely send information to a therapist dashboard. Further, the API may receive information for a database.

For example, the input/output interface 150 may play a role of an interface for delivering an instruction or data input from a user or a different external device(s) to the different constituent elements of the electronic device 101. Further, the input/output interface 150 may output an instruction or data received from the different constituent element(s) of the electronic device 101 to the different external device.

The display 160 may include various types of displays, for example, an AR/VR display, Liquid Crystal Display (LCD) display, a Light Emitting Diode (LED) display, an Organic Light-Emitting Diode (OLED) display, a MicroElectroMechanical Systems (MEMS) display, or an electronic paper display. The display 160 may display, for example, a variety of contents (e.g., text, image, video, icon, symbol, etc.) to the user. The display 160 may include a touch screen. For example, the display 160 may receive a touch, gesture, proximity, or hovering input by using a stylus pen or a part of a user's body. In an example, the display 160 may be configured for emitting scenes (e.g., AR/VR experiences).

The communication interface 170 may establish, for example, communication between the electronic device 101 and the external device (e.g., a 1st external electronic device 102, a 2nd external electronic device 104, or a server 106). For example, the communication interface 170 may communicate with the external device (e.g., the 2nd external electronic device 104 or the server 106) by being connected to a network 162 through wireless communication or wired communication. External devices, may for example, comprise external data sources (e.g., external data sources 308). External data sources 308 may be in communication with various other devices.

For example, as a cellular communication protocol, the wireless communication may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. Further, the wireless communication may include, for example, a near-distance communication 164. The near-distance communication 164 may include, for example, at least one of Wireless Fidelity (WiFi), Bluetooth, Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. The wired communication may include, for example, at least one of Universal Serial Bus (USB), High Definition Multimedia Interface (HDMI), Recommended Standard-232 (RS-232), power-line communication, Plain Old Telephone Service (POTS), and the like. The network 162 may include, for example, at least one of a telecommunications network, a computer network (e.g., LAN or WAN), the internet, and a telephone network.

Each of the 1st and 2nd external electronic devices 102 and 104 may be the same type or different type of the electronic device 101. In an embodiment, either the 1^{st} or 2^{nd} electronic devices 101, 102, or 104 may be an AR/VR device. The AR/VR device may comprise one or more light emitting diodes (LED), one or more liquid crystal displays (LCD), one or more Cold Cathode Fluorescent Lamps (CCFL), combinations thereof, and the like. The AR/VR device may be configured for outputting triggers or other stimuli. The AR/VR device may be configured for emitting light, displaying audio, video, or image content or the like. The AR/VR device may be configured to receive an input such as a voice input or other auditory input, visual input, data and the like. The application program 147 may be configured to communicate with the electronic device 102 via the network 164 to control the output of the AR/VR device or the input received by the AR/VR device.

According to one exemplary embodiment, the server 106 may include a group of one or more servers. According to various exemplary embodiments, all or some of the operations executed by the electronic device 101 may be executed in a different one or a plurality of electronic devices (e.g., the electronic device 102 or 104 or the server 106). According to one exemplary embodiment, if the electronic device 101 needs to perform a certain function or service either automatically or at a request, the electronic device 101 may request at least some parts of functions related thereto alternatively or additionally to a different electronic device (e.g., the electronic device 102 or 104 or the server 106) instead of executing the function or the service autonomously. The different electronic device (e.g., the electronic device 102 or 104 or the server 106) may execute the requested function or additional function, and may deliver a result thereof to the electronic device 101. The electronic device 101 may provide the requested function or service either directly or by additionally processing the received result. For this, for example, a cloud computing, distributed computing, or client-server computing technique may be used.

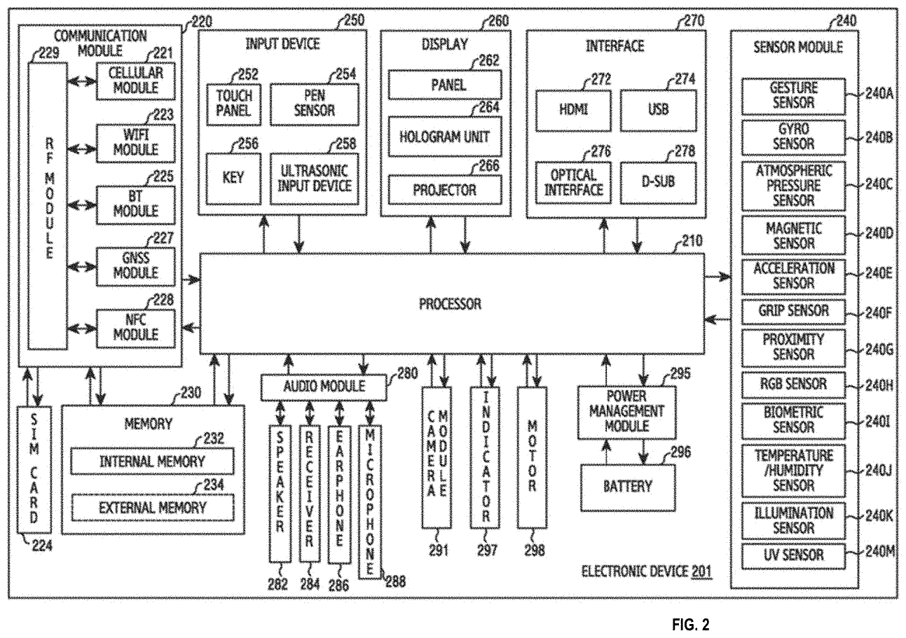

FIG. 2 is a block diagram of an electronic device according to various exemplary embodiments. The electronic device of the FIG. 2 may comprise, for example, the electronic device of FIG. 1. An electronic device 201 may include, for example, all or some parts of the electronic device 101 of FIG. 1. The electronic device 201 may include one or more processors (e.g., Application Processors (APs)) 210, a communication module 220, a subscriber identity module 224, a memory 230, a sensor module 240, an input unit 250, a display 260, an interface 270, an audio module 280, a camera unit 291, a power management module 295, a battery 296, an indicator 297, and a motor 298.

The processor 210 may control a plurality of hardware or software constituent elements connected to the processor 210 by driving, for example, an operating system or an application program, and may process a variety of data including multimedia data and may perform an arithmetic operation. The processor 210 may be implemented, for example, with a System on Chip (SoC). According to one exemplary embodiment, the processor 210 may further include a Graphic Processing Unit (GPU) and/or an Image Signal Processor (ISP). The processor 210 may include at least one part (e.g., a cellular module 221) of the aforementioned constituent elements of FIG. 2. The processor 210 may process an instruction or data, which is received from at least one of different constituent elements (e.g., a non-volatile memory), by loading it to a volatile memory and may store a variety of data in the non-volatile memory.

The communication module 220 may have a structure the same as or similar to the communication interface 170 of FIG. 1. The communication module 220 may include, for example, the cellular module 221, a Wi-Fi module 223, a BlueTooth (BT) module 225, a GNSS module 227 (e.g., a GPS module, a Glonass module, a Beidou module, or a Galileo module), a Near Field Communication (NFC) module 228, and a Radio Frequency (RF) module 229.

The cellular module 221 may provide a voice call, a video call, a text service, an internet service, or the like, for example, through a communication network. According to one exemplary embodiment, the cellular module 221 may identify and authenticate the electronic device 201 in the communication network by using the subscriber identity module (e.g., a Subscriber Identity Module (SIM) card) 224. According to one exemplary embodiment, the cellular module 221 may perform at least some functions that can be provided by the processor 210. According to one exemplary embodiment, the cellular module 221 may include a Communication Processor (CP).

Each of the WiFi module 223, the BT module 225, the GNSS module 227, or the NFC module 228 may include, for example, a processor for processing data transmitted/received via a corresponding module. According to a certain exemplary embodiment, at least some (e.g., two or more) of the cellular module 221, the WiFi module 223, the BT module 225, the GPS module 227, and the NFC module 228 may be included in one Integrated Chip (IC) or IC package.

The RF module 229 may transmit/receive, for example, a communication signal (e.g., a Radio Frequency (RF) signal). The RF module 229 may include, for example, a transceiver, a Power Amp Module (PAM), a frequency filter, a Low Noise Amplifier (LNA), an antenna, or the like. According to another exemplary embodiment, at least one of the cellular module 221, the WiFi module 223, the BT module 225, the GPS module 227, and the NFC module 228 may transmit/receive an RF signal via a separate RF module.

The subscriber identity module 224 may include, for example, a card including the subscriber identity module and/or an embedded SIM, and may include unique identification information (e.g., an Integrated Circuit Card IDentifier (ICCID)) or subscriber information (e.g., an International Mobile Subscriber Identity (IMSI)).

The memory 230 (e.g., the memory 130) may include, for example, an internal memory 232 or an external memory 234. The internal memory 232 may include, for example, at least one of a volatile memory (e.g., a Dynamic RAM (DRAM), a Static RAM (SRAM), a Synchronous Dynamic RAM (SDRAM), etc.) and a non-volatile memory (e.g., a One Time Programmable ROM (OTPROM), a Programmable ROM (PROM), an Erasable and Programmable ROM (EPROM), an Electrically Erasable and Programmable ROM (EEPROM), a mask ROM, a flash ROM, a flash memory (e.g., a NAND flash memory, a NOR flash memory, etc.), a hard drive, or a Solid State Drive (SSD)).

The external memory 234 may further include a flash drive, for example, Compact Flash (CF), Secure Digital (SD), Micro Secure Digital (Micro-SD), Mini Secure digital (Mini-SD), extreme Digital (xD), memory stick, or the like. The external memory 234 may be operatively and/or physically connected to the electronic device 201 via various interfaces.

The sensor module 240 may measure, for example, physical quantity or detect an operational status of the electronic device 201, and may convert the measured or detected information into an electric signal. The sensor module 240 may include, for example, at least one of a gesture sensor 240A, a gyro sensor 240B, a pressure sensor 240C, a magnetic sensor 240D, an acceleration sensor 240E, a grip sensor 240F, a proximity sensor 240G, a color sensor 240H (e.g., a Red, Green, Blue (RGB) sensor), a bio sensor 240I, a temperature/humidity sensor 240J, an illumination sensor 240K, an Ultra Violet (UV) sensor 240M, an ultrasonic sensor 240N, and an optical sensor 240P. According to one exemplary embodiment, the optical sensor 240P may detect ambient light and/or light reflected by an external object (e.g., a user's finger. etc.), and which is converted into a specific wavelength band by means of a light converting member. Additionally or alternatively, the sensor module 240 may include, for example, an E-nose sensor, an ElectroMyoGraphy (EMG) sensor, an ElectroEncephaloGram (EEG) sensor, an ElectroCardioGram (ECG) sensor, an Infrared (IR) sensor, an iris sensor, and/or a fingerprint sensor. The sensor module 240 may further include a control circuit for controlling at least one or more sensors included therein. In a certain exemplary embodiment, the electronic device 201 may further include a processor configured to control the sensor module 204 either separately or as one part of the processor 210, and may control the sensor module 240 while the processor 210 is in a sleep state.

The input device 250 may include, for example, a touch panel 252, a (digital) pen sensor 254, a key 256, or an ultrasonic input device 258. The touch panel 252 may recognize a touch input, for example, by using at least one of an electrostatic type, a pressure-sensitive type, and an ultrasonic type. In addition, the touch panel 252 may further include a control circuit. The touch penal 252 may further include a tactile layer and thus may provide the user with a tactile reaction.

The (digital) pen sensor 254 may be, for example, one part of a touch panel, or may include an additional sheet for recognition. The key 256 may be, for example, a physical button, an optical key, a keypad, or a touch key. The ultrasonic input device 258 may detect an ultrasonic wave generated from an input means through a microphone (e.g., a microphone 288) to confirm data corresponding to the detected ultrasonic wave.

The display 260 (e.g., the display 160) may include a panel 262, a hologram unit 264, or a projector 266. The display 260 may comprise an AR/VR display. The panel 262 may include a structure the same as or similar to the display 160 of FIG. 1. The panel 262 may be implemented, for example, in a flexible, transparent, or wearable manner. The panel 262 may be constructed as one module with the touch panel 252. According to one exemplary embodiment, the panel 262 may include a pressure sensor (or a force sensor) capable of measuring strength of pressure for a user's touch. The pressure sensor may be implemented in an integral form with respect to the touch panel 252, or may be implemented as one or more sensors separated from the touch panel 252.

The hologram unit 264 may use an interference of light and show a stereoscopic image in the air. The projector 266 may display an image by projecting a light beam onto a screen. The screen may be located, for example, inside or outside the electronic device 201. According to one exemplary embodiment, the display 260 may further include a control circuit for controlling the panel 262, the hologram unit 264, or the projector 266.

The interface 270 may include, for example, a High-Definition Multimedia Interface (HDMI) 272, a Universal Serial Bus (USB) 274, an optical communication interface 276, or a D-subminiature (D-sub) 278. The interface 270 may be included, for example, in the communication interface 170 of FIG. 1. Additionally or alternatively, the interface 270 may include, for example, a Mobile High-definition Link (MHL) interface, a Secure Digital (SD)/Multi-Media Card (MMC) interface, or an Infrared Data Association (IrDA) standard interface.

The audio module 280 may bilaterally convert, for example, a sound and electric signal. At least some constituent elements of the audio module 280 may be included in, for example, the input/output interface 150 of FIG. 1. The audio module 280 may convert sound information which is input or output, for example, through a speaker 282, a receiver 284, an earphone 286, the microphone 288, or the like.

The camera module 291 is, for example, a device for image and video capturing, and according to one exemplary embodiment, may include one or more image sensors (e.g., a front sensor or a rear sensor), a lens, an Image Signal Processor (ISP), or a flash (e.g., LED or xenon lamp).

The power management module 295 may manage, for example, power of the electronic device 201. According to one exemplary embodiment, the power management module 295 may include a Power Management Integrated Circuit (PMIC), a charger Integrated Circuit (IC), or a battery fuel gauge. The PMIC may have a wired and/or wireless charging type. The wireless charging type may include, for example, a magnetic resonance type, a magnetic induction type, an electromagnetic type, or the like, and may further include an additional circuit for wireless charging, for example, a coil loop, a resonant circuit, a rectifier, or the like. The battery gauge may measure, for example, residual quantity of the battery 296 and voltage, current, and temperature during charging. The battery 296 may include, for example, a rechargeable battery and/or a solar battery.

The indicator 297 may display a specific state, for example, a booting state, a message state, a charging state, or the like, of the electronic device 201 or one part thereof (e.g., the processor 210). The motor 298 may convert an electric signal into a mechanical vibration, and may generate a vibration or tactile or haptic effect. Although not shown, the electronic device 201 may include a processing device (e.g., a GPU) for supporting a mobile TV. The processing device for supporting the mobile TV may process media data conforming to a protocol of, for example, Digital Multimedia Broadcasting (DMB), Digital Video Broadcasting (DVB), MediaFlo™, or the like.

Each of the constituent elements described in the present document may consist of one or more components, and names thereof may vary depending on a type of an electronic device. The electronic device 101 according to various exemplary embodiments may include at least one of the constituent elements described in the present document. Some of the constituent elements may be omitted, or additional other constituent elements may be further included. Further, some of the constituent elements of the electronic device according to various exemplary embodiments may be combined and constructed as one entity, so as to equally perform functions of corresponding constituent elements before combination.

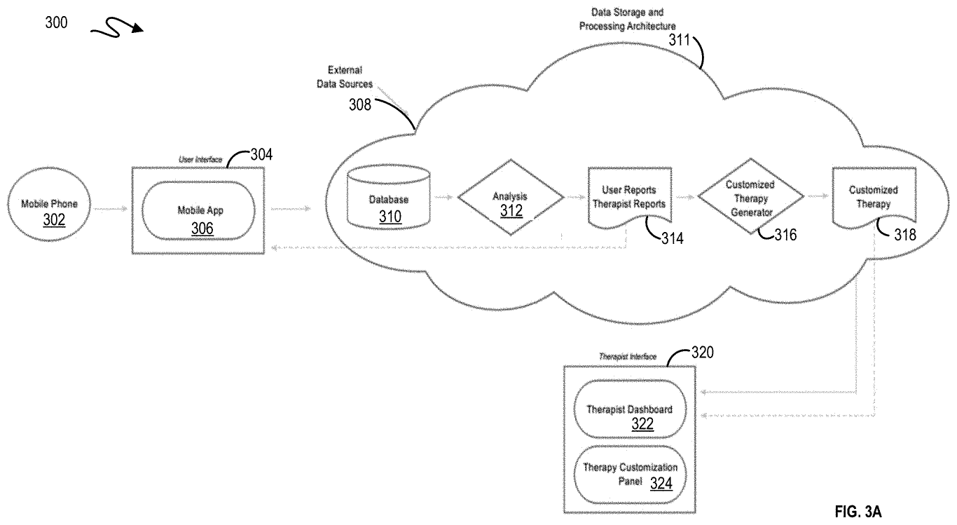

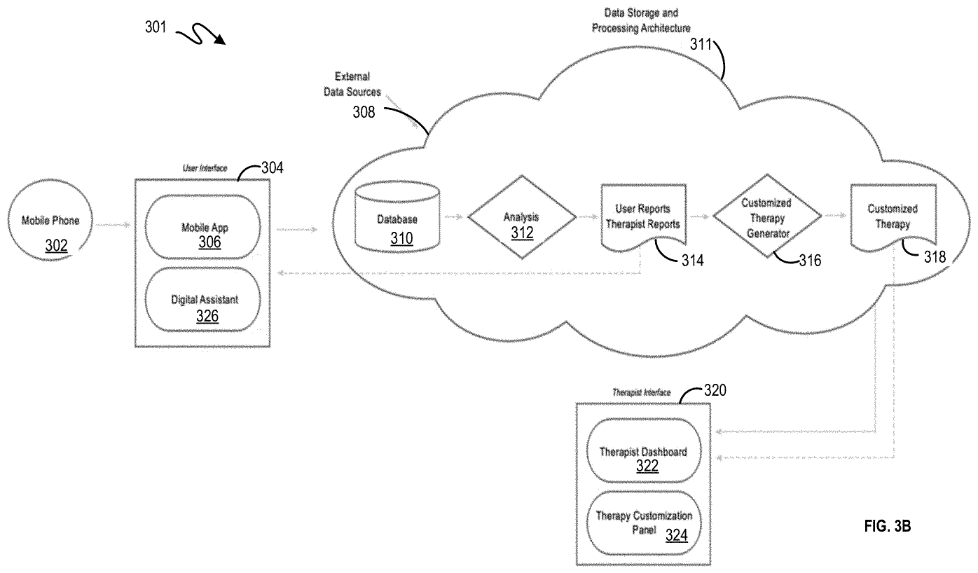

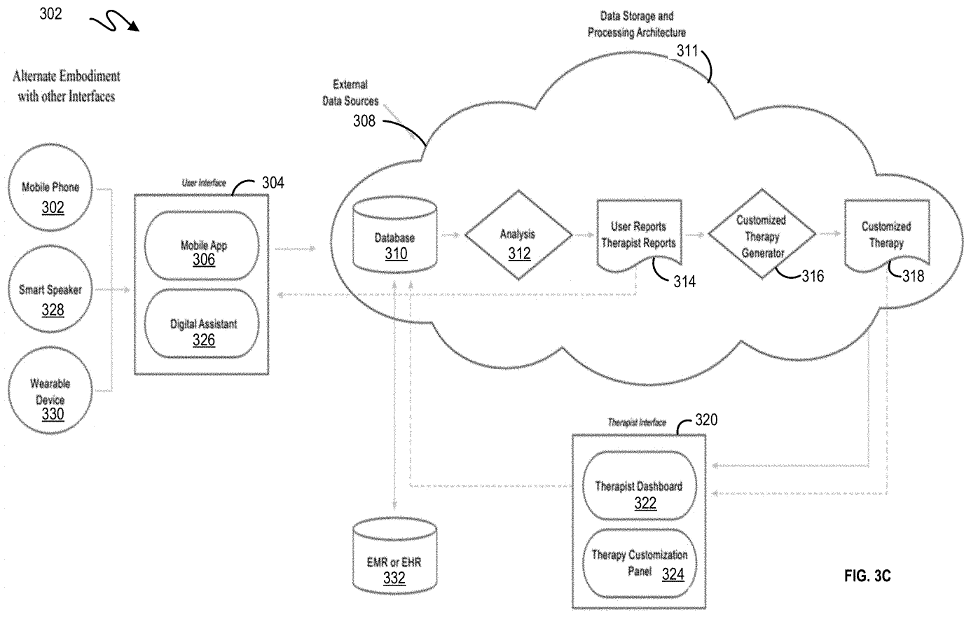

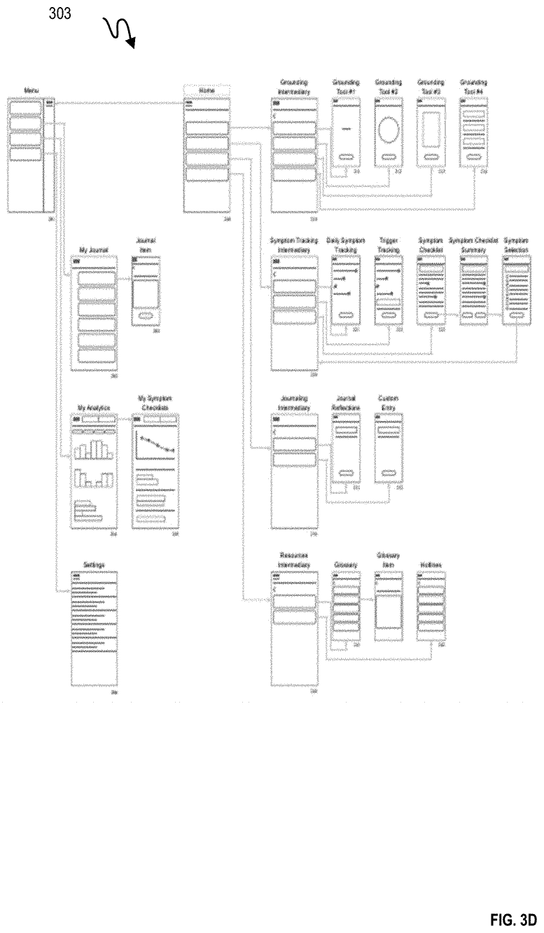

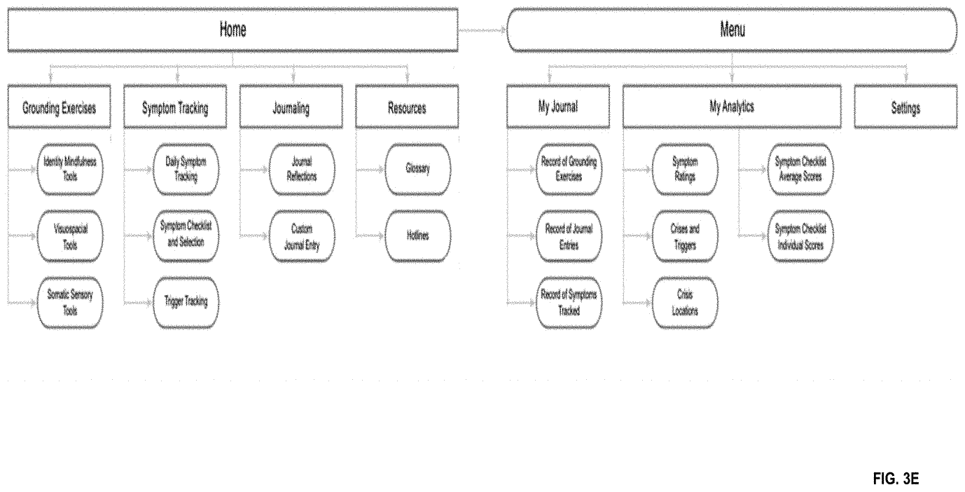

FIG. 3A illustrates an example system 300 according to various embodiments of the present disclosure. The system 300 may comprise various components which may be in communication with some or other or all components. For example, the system 300 may comprise a user device 302 (for example, a mobile phone), such as the electronic device 101 and/or the electronic device 201. The system 300 may comprise an application 306 (for example a mobile application) which may be disposed on a user device and displayed on an interface 304. The system may further comprise a database 310. The database 310 may be an electronic database. For example, the database 310 may comprise electronic medical records, health records, identifiers and the like. The database 310 may further comprise AR/VR data. The database 310 may store image or video or audio content or combinations thereof. For example, the database 310 may store AR/VR experiences. The system may comprise an analysis engine 312. The analysis engine 312 may analyze data in the database 310 to generate reports 314 such as user reports and or therapist reports. The system may further comprise a customized therapy generator 316 which may generate customized therapy 318. Further, the customized therapy generator 316 may comprise an AR/VR experience generator. The system component configuration 300 may also include but not be limited to a therapist interface 320 which may comprise a therapist dashboard 322 and/or a therapy customization panel 324. Symptom tracking and journaling data may be collected through logs from the use of a user interface, and from manual entries from the user that can be collected anytime and anywhere. Logging may be automatic or manual. A user may interact with a user interface to log certain information. Information entered or logged by the user may be transmitted to a database, for example the database 310. Certain information may be logged automatically, for example, the time, date, location, and duration and/or frequency of use of the application. Data which may be logged manually may, for example, include recording experiences or thoughts or other, user-specific data. Further, user identifiers can be logged, stored, and associated with other various data including therapeutic functions, diagnostic functions, biometric information and the like. Other data may also be logged, for example, diagnostic data such as symptoms, user characteristics and other data as well as biometric data such as heart rate or other biometric information. Thus providing a unique dataset with actual information on symptoms, location, triggers, etc. that are not available when relying on the user's memory. This data provides unique information that may, alone or processed with additional data sources, inform: treatment decisions, evaluation of treatment, status of the disorder, changes in symptoms, and the creation of personalized treatments. Treatment decisions may comprise diagnostic or therapeutic functions such custom therapy experiences.

The data may be extrapolated and/or analyzed to provide insights to users. Such analysis may be performed by an analysis engine such as analysis engine 312. Analysis engine 312 may comprise software or an algorithm which may analyze data. Exemplary algorithms include but are not limited to location algorithm 910, time of day algorithm 912, or audio processing algorithm 914 or video or image processing algorithms. Such analysis may comprise creating associations between data (for example, symptom tracking and journaling), performing mathematical operations or other analytical functions on data, for example, matching symptoms to a diagnosis.

Further, a user may interact with a user interface 304 to participate in experiences such as AR/VR experiences. Additionally, a user may interact with a user interface 304 to participate in grounding exercises such as color-finding, color-matching, breathing exercises, meditative exercises, tactile or haptic exercises and other exercises and the like.

The analysis engine 312 may gather information from the user's journal entries and use those insights to modify grounding techniques to best support the user. For example, a user may submit a pre-exercise symptom rating, complete an exercise or experience, and submit a post-exercise or post-experience symptom rating. This data can be collected and analyzed to provide optimal therapeutic or diagnostic functions. Decision trees and machine learning (ML) algorithms let the analysis engine 312 suggest relevant journal entries, and use the expanding set of entries to personalize its grounding techniques. Because users can experience varying dissociative symptoms in different times and settings, personalization is critical for generating an impactful therapeutic experience. By interacting with patients during dissociative episodes, the system automatically tracks details about the patient such as time and location data as well as further information stored in a journal entry and shares information with approved therapists. This data may be used to generate exercises or experiences. For example, the system may log location data and using that data, may retrieve additional data such as maps or images to be displayed to the user. Because dissociation includes depersonalizing and amnesic aspects, manually tracking such symptoms is difficult and does not always yield reliable information. By automatically tracking insights such as symptoms, triggers, and preferred grounding methods, the system provides therapists with accurate and actionable information which may be provided to users and therapists.

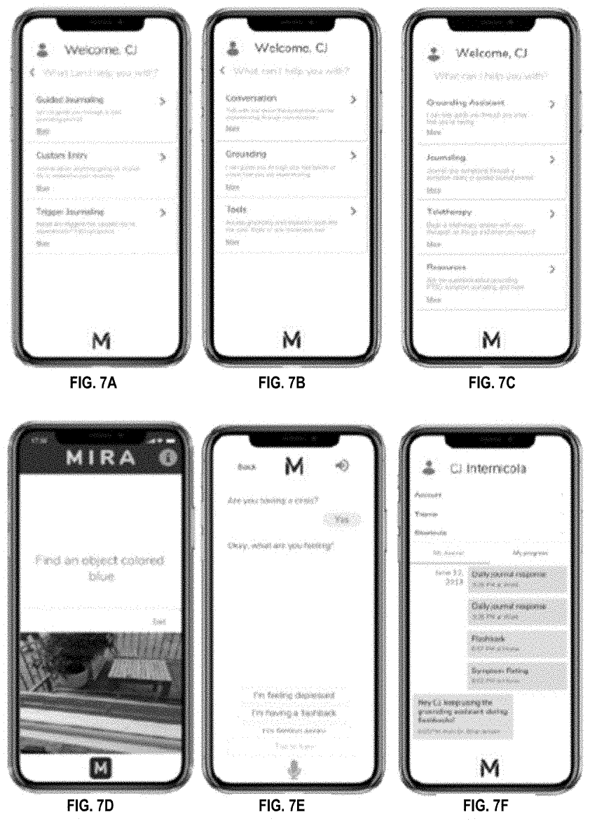

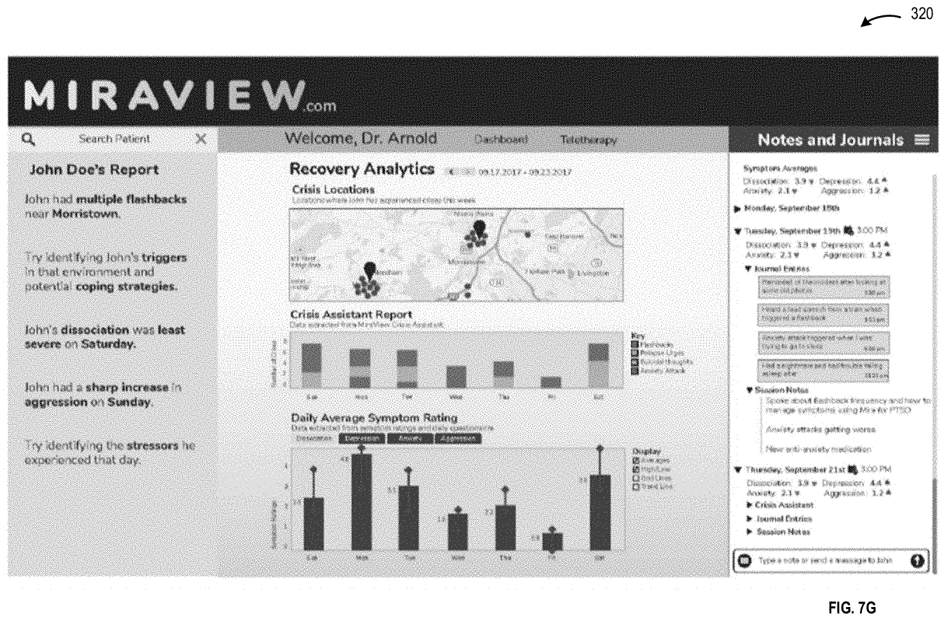

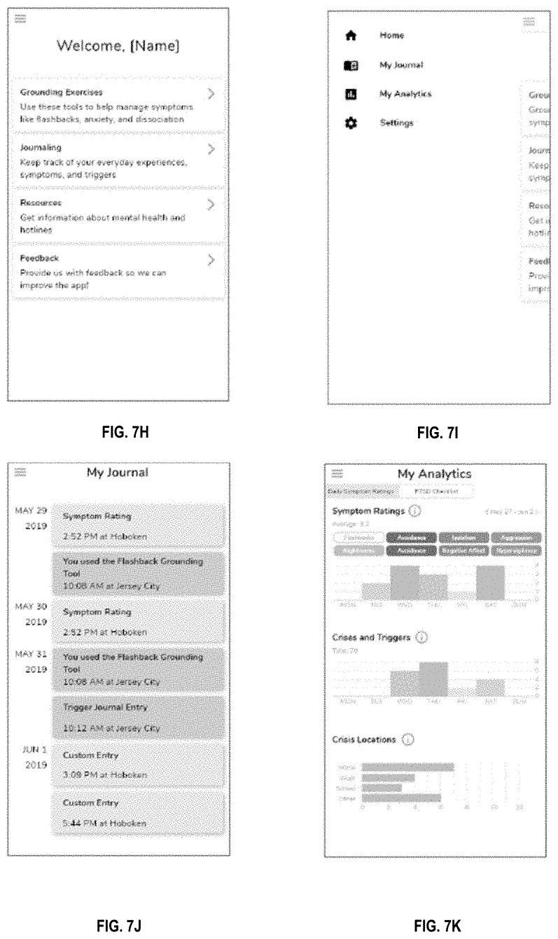

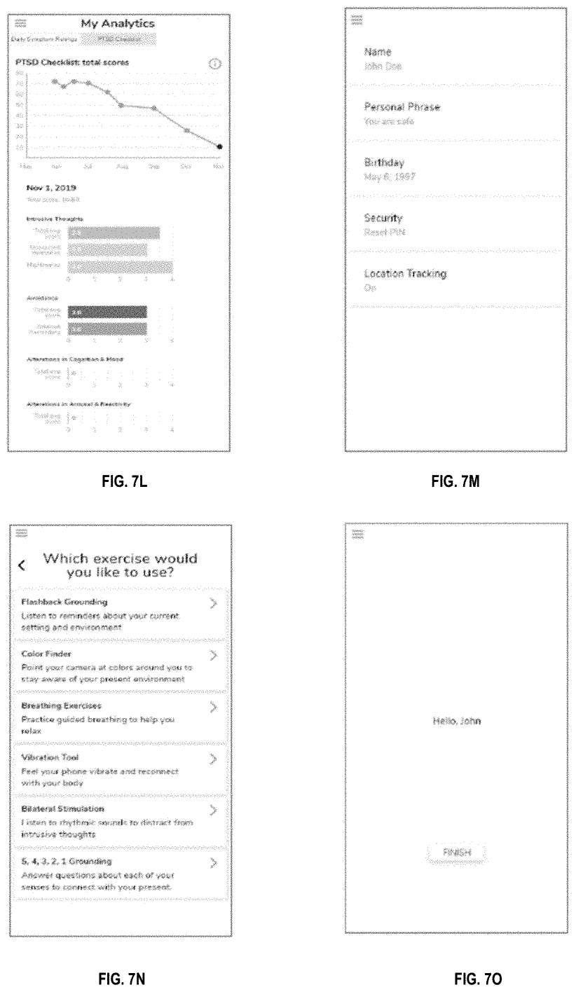

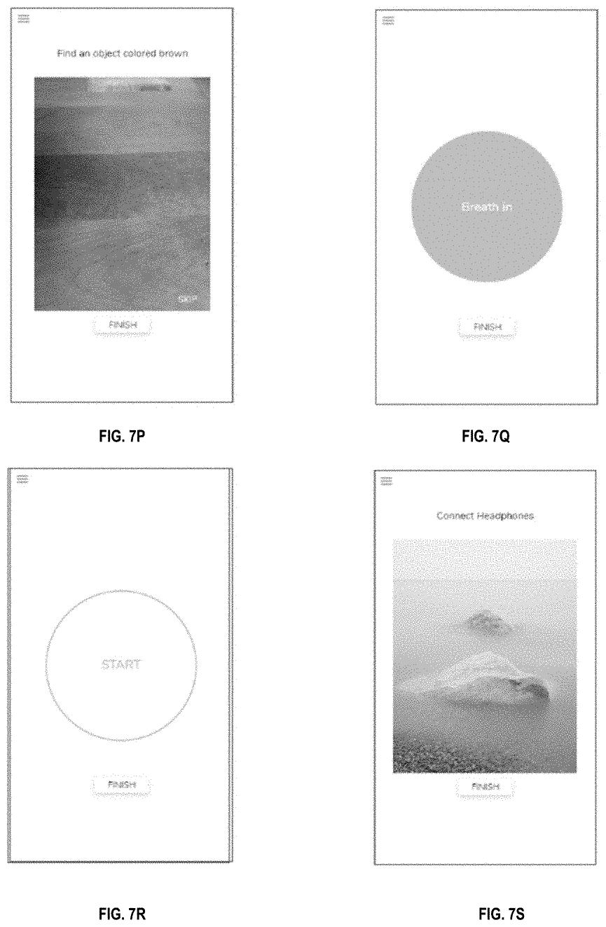

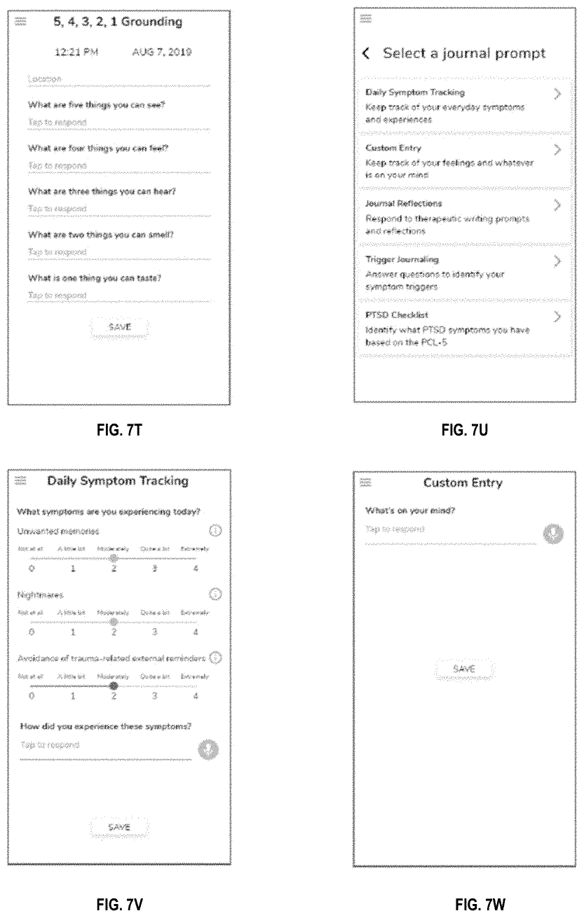

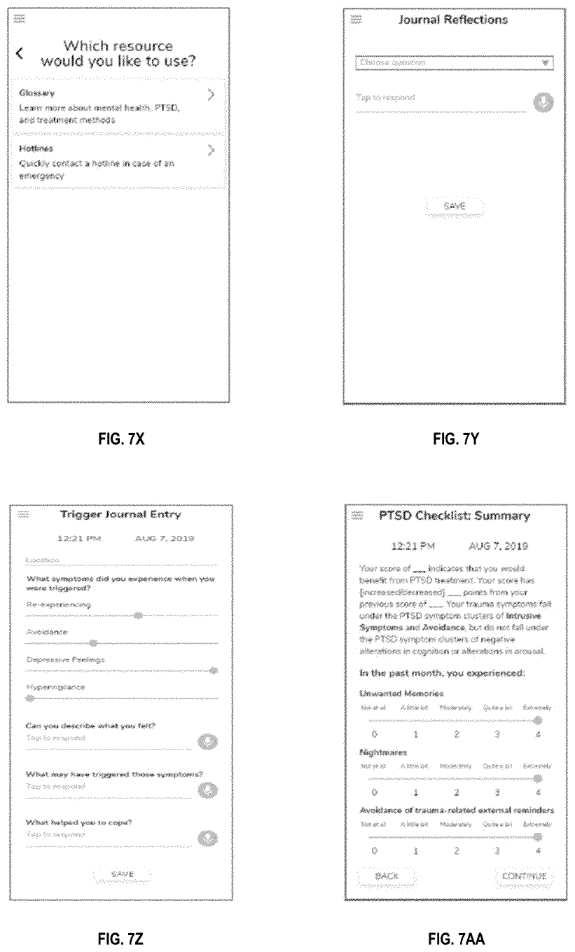

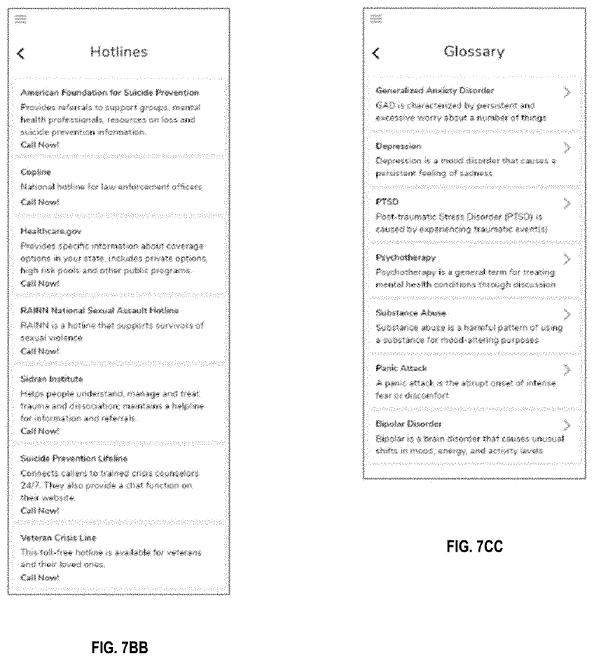

Therapists may be mental health professionals such as psychologists, psychiatrists, and social workers, etc. who treat people with mental health disorders such as the ones experienced by the users. Therapists may use a therapist interface 320 (e.g., the therapist interface of FIG. 7G) to see and access information regarding a client's symptoms, progress and experiences. Information and results from reports 314 (e.g., therapist reports or user reports) may be displayed on the therapist dashboard 322, thereby providing unique insights from the data collected through the user interface and possibly combined with data from other sources. Therapists may use this information to evaluate the client's condition, and to inform treatment decisions, and therapy sessions. The therapist dashboard 322 may allow therapists or other users to view patient insights. This interface may receive patient information (such as patient report files), and may visualize such information to supplement evidence-based therapy methods. Patient report files may comprise user identifiers as well as other information such as diagnostic or therapeutic data, for example diagnostic or therapeutic histories. A diagnostic or therapeutic history may recorded by the application be recording user inputs and/or activities. Among other uses, this dashboard may help therapists select discussion topics, identify, symptom triggers, notice changes in patients' emotional states, and develop treatment plans. Therapists may execute diagnostic or therapeutic functions by way of the therapy customization panel 332.

Therapists may use the therapist interface 320 to visualize and analyze a client population. This information can help identify best practices, and the most effective therapies. It can also help identify new treatment options, for example for new cases that are similar to previous clients. The therapist interface 320 may be an interface as depicted in FIG. 7G. The therapist interface 320 may display data as well as facilitate an interaction between a user and data.

Therapists may use the therapist interface 322 and the therapy customization panel 324 to deliver Exposure Therapy. For each client who is a user, customized AR/VR Experiences may be provided by the custom therapy generator 316 which may comprise an AR/VR generator for each particular client. The customized AR/VR experience provides a personalized experience based on previous triggers and environments that have been identified through the data collected. During an Exposure Therapy session, therapists may deliver the AR/VR experience to the client in a controllable, confined virtual space. Exposure Therapy helps sufferers develop normal reactions to stimuli that triggers their symptoms. Triggers may be different for different users. Therefore, identification and exposure to the actual personal triggers, as provided by the AR/VR Experience, is important for successful treatment.

The customized therapy generator 316 may generate experiences and/or exercises. Experiences may comprise recreations of circumstances which caused a user to experience a flashback or some other symptom. For example, an experience may comprise a recreation of a car backfiring or a train passing. Such experiences can be displayed using AR and/or VR. In a further example, an experience may comprise a recreation of a scene, for example a location like a neighborhood or a scene such as an interaction between individuals. The customized therapy generator 316 may generate customized therapies based on input received from a user or a therapist or both. For example, the customized therapy generator may receive location information from a user device and may retrieve location information from a database or from a mapping service or the internet. The custom therapy generator may combine data from various sources to generate a customized therapy experience, for instance recreating a scene in a neighborhood. For example, the custom therapy generator 316 may receive data from database 310 such as a scene and overlay the scene with audio captured by a user. Further, the customized therapy generator 316 may recreate scenes like a construction site or some other scene. These scenes may be rendered in AR or VR. They may be fictional or no-fictional. Further, the customized therapy generator 316 may receive input such as data, for example diagnostic or therapeutic data like symptom ratings or temporal or special information. In a further additional example, the customized therapy generator 316 may gather data from a databased, the internet, or another source. The data may comprise visual, audio, or other data. The customized therapy generator 316 may generate a scene using the data. A user and/or therapist may interact with the scene via a user interface 304.

The custom therapy generator 316 may be in communication with a database such as database 310. The database may store pre-programmed or pre-loaded experiences or exercises. For example, the database 310 may store beach scenes or field scenes. In another example, the database 310 may comprise audio-visual scenes such as a train passing or a construction site scene.

Further, the custom therapy generator 316 may create custom scenes or experiences. The custom therapy generator 316 may also use template, pre-loaded scenes or experiences. For example, a user may submit location and temporal data such as a street corner and time of day at which the user experienced a symptom, for example a flashback. For example, a user may log (manually or automatically) a time and location at which the user experienced a symptom. The application 306 may, by using the location data, retrieve data about the location such as a map or images or a street view. Based on information submitted by the user, for example, a description of a scene such as a construction site, the custom therapy generator 316 may generate a scene by outputting visual data such as an image overlayed with audio data such as a sound recording of a jack-hammer or the like, and my further overlay the scene with the retrieved location images. These examples are in no way meant to be limiting.

Further, the user may contribute to the creation of the experience by capturing information such as location or temporal information, for instance a video or a photo or audio recording. For example, a user may, when experiencing a symptom or a flashback, record a scene by taking a video or a picture. The user may upload that scene to the database 310. The customized therapy generator 316 may, based on the data, generate an experience.

The custom therapy generator 316 may create personalized AR/VR experiences based on each individual patient's Patient Report Files, To execute this function, the system may first identify a number of data points that are associated with the user's triggers. Such information may be automatically gathered when the patient uses the digital assistant 326 for the purpose of grounding or symptom support. The digital assistant 326 may be manifest in a mobile app or other software and may interact with a user or therapist by way of a user device such as a computer, smart phone, wearable device (e.g., wearable device 330) or a voice enable device (e.g., smart speaker 328). These data points may include the time, location, and statements recorded during the session. Using such data, this component may create AR/VR experiences that mimic the settings that triggered states of elevated dissociation, or anxiety or other symptoms in a particular user. This component creates AR/VR Exposure Therapy Experiences, and saves such experiences, along with their individual elements, in an AR/VR Therapy Database, for example, database 310. AR/VR Exposure Therapy Experiences may visualize real-world locations (either indoors or outdoors), depict different times of day via lighting, and incorporate background sounds related to the real world locations. Such experiences are powerful tools for administering Exposure Therapy.

The personalized AR/VR Exposure Therapy Experiences may be based on a patient's unique data, for example a user identifier or a device identifier. As a patient uses the digital assistant 326, the application 306 or the digital assistant 326 may track a series of key data points associated with the patient's triggers. Such data points are compiled into Patient Report Files and securely shared over the back-end Data Sharing Architecture into a Patient Report Database. Once reports are shared to the database 310, The AR/VR Experience Generator uses a series of algorithms to create VR elements and store them in a AR/VR Therapy Database. The first of these algorithms is a Location Algorithm 910 that matches GPS coordinates with location images from a Google Maps API. A Brightness Algorithm then uses date and time data to determine the appropriate brightness of the sky depicted in the AR/VR Exposure Therapy Experience. An audio processing algorithm may then estimates the type of background noise and the intensity of such noise that the patient may have heard when he/she activated the digital assistant 326. Once the algorithm identifies the location, brightness, and noise details associated with a patient's trigger, it saves the individual elements in an AR/VR Therapy Database, and gathers places them into an AR/VR Exposure Therapy Generation Schema. This schema takes the AR/VR elements it compiles them into unique AR/VR Exposure Therapy Experiences. Those compiled experiences may also be stored in the VR Therapy Database.

The digital assistant 326 may receive an input from a user or a therapist. In response to receiving the input, the digital assistant 326 may transmit data or output content such as a stimuli, trigger, or experience. The digital assistant 326 may ask the user to confirm they are having a crisis and then differentiate between the type of crisis that the user is having. The digital assistant 326 may be configured to determine whether or not the patient is having a crisis by voice commands and by doing so has the ability to indicate the mental state of the patient. The digital assistant 326 may be configured to determine whether or not the patient is having a crisis based on biometric data such as a voice print, pulse, pupil dilation and the like as measured by a device. The digital assistant 326 may also be configured to store and access previously recorded entries made from using a feature within the mobile application. The digital assistant 326 may be further configured to facilitate journaling. Additionally, the digital assistant 326 may be configured to facilitate teletherapy. Further, the digital assistant may be configured to facilitate symptom rating.

The digital assistant 326 may be configured for self-managing symptom as well as accurately tracking progress. While using the digital assistant 326, users can activate grounding technique modules, and/or journal their experiences, thoughts, or symptoms at any time. Their actions may be stored as Patient Report Files, which users like sufferers or therapists can view at any time. The digital assistant 326 may push Patient Report Files through a back-end Data Sharing Architecture (for example, data storage and processing architecture 311), embodied in, for example, an AWS hosted and HIPAA compliant database, such as DynamoDB, where such data may be aggregated and shared to the therapist dashboard as well as the customized therapy generator 316 and/or the AR/VR Experience Generator. When the digital assistant 326 is properly connected to the therapist dashboard, therapists are also be able to view Patient Report Files, and can create “Therapist Entries,” which may be shared to a patient's digital assistant 326 and viewable in the Patient Report Files. Patients are also able to delete information in the Patient Report Files, as well as edit dates, and content of individual entries. The digital assistant's 326 user interface (UI) may be launched within the app by clicking a designated icon, or other designated module buttons. A UI may comprise a vocal UI (VUI). The digital assistant's 326 UI may be a conversational bot, which may be configured to be implemented with AWS Lex. Lex uses advanced Natural Language Processing (NLP), and Machine Learning (ML) over time to learn similar words that can launch a desired utterance. The UI may be able support users with a number of different features. For example, users may manually record information by saying “Log a new journal entry.” The digital Aassistant's 326 UI can also prompt the user with follow-up questions designed to identify symptoms and make decisions on what features it predicts the user would benefit from using at that time. Users are also able to launch various grounding tools through the UI. After using a grounding tool in the digital assistant 326, the system may automatically log that interaction in the Patient Report File. This entry may automatically note certain data points, for example location or temporal data or biometric data, and the user may edit the entry to record more details about the situation which triggered their symptoms. One UI compatible grounding tool incorporated in the digital assistant 326 may be a Script Reader tool, which reads off a user-specific script designed to ground that user. The script includes details such as the user's current location, current time, age, and name. The script also incorporates statements based on recent symptom data gathered from other tools. Other compatible grounding tools include a game that prompts the user to point their device camera at specific colors, a tool that prompts the user to record things they can identify with their live senses, a tool that uses vibrations, a tool that uses binaural sounds, a tool that uses a moving red dot, and a tool that guides users through breathing exercises.

In one embodiment, the user interface 304 of this embodiment may comprise an application 306 such as a mobile application that may be accessed through a user device 302 which may be a mobile phone or the like. The user device (for example, mobile phone 302) may be a computer or a mobile device such as mobile phone, computer, wearable, or any other personal device that allows for user interaction, either through a graphic, voice, sensory or neural interface. In this embodiment, the application 306 may comprise four modules which may be accessible from the home screen and several modules accessible form a menu but other arrangements of the modules are also suitable. The modules in this example that may be accessible from the Home Screen are: Grounding exercises module, Symptom Tracking module, Journaling module, and Resources module. The modules in this embodiment which may be accessible from the Menu are: My Journal module, My Analytics module, and Settings. The Menu may appear in all screens or some screens.

In one embodiment, the Grounding exercises module may provide a series of grounding exercises and tools that the user can choose from. The number of grounding tools or exercises may vary, whether within this module or in another configuration of the User interface. Grounding exercises can be classified as: identity mindfulness tools, visuospatial tools, and somatic sensory tools. Examples of grounding exercises are:

a) Identity mindfulness tool: a script showing in the screen and audible through the device speakers, reminding the user of the present, such as their name, age, present location and time. The script may repeat until the user stops it or closes the application,

b) Visuospatial tool: a game-like activity prompting users point the build-in camera of their mobile phone at specific colors in their surroundings.

c) Somatic sensory tool: a guided breathing exercise, a sequential vibration of the mobile phone being held by the user.

In an embodiment, The Symptom Tracking module may provide screens to record or rate symptoms, experiences, and thoughts. In this embodiment, the Symptom Tracking Module includes the following components, but other arrangements are suitable:

a) Daily Symptoms Tracking, for daily recording of overall symptoms

b) Trigger Tracking, to record information on triggers to symptomatic events

c) Symptom Checklist and Selection, includes standardized checklists of symptoms and rating to identify main symptoms and choose which ones to track through the Daily Symptoms Tracking.

The Journaling module may provide tools for general recording of thoughts, experiences, feelings, etc. that the user wants to remember or simply express. This embodiment may comprise a Reflections module, to record any thoughts. The Journaling module may further comprise a Custom Journal Entry.

The Resources module may provide information on mental health and other resources to seek help. The resources module may further comprise a glossary of mental health terms as well as a list of Hotlines, but other information and resources are suitable. The Hotlines may be provided so that a call can be made directly by tapping the phone number provided in the list.

The exemplary embodiment may also contain the description for the Data Storage and Processing Architecture, for example data storage and processing architecture 311. Data collected through the user interface may be stored in a database, for example database 310. Data storage, processing, management, and sharing may be compliant with privacy and HIPAA regulations. Data may be summarized and analyzed to produce user reports and therapist reports, for example user reports and therapist reports 314. User reports may include but are not limited to: a) summary presentations of the information provided through the Symptom Tracking Module, b) results from standard rating scales over time, and c) other user information and data available. Therapist reports may include the same information as the user reports and in addition include information across Users who are clients of the therapist. The user interface 304 may be the source of data, but other sources are suitable. These other data sources may be connected directly to the database 310 or provide data through the user interface 304. In this example, data analytics may be described as summaries, but other analytical techniques are suitable, in particular when adding other data or data sources. User reports and Therapist Reports in this embodiment may correspond to the data provided through the user interface 304 as described above. With the use of other data sources and analytic techniques, other summaries and analysis may be suitable and may be included in the User reports and in the Therapist Reports.

User Reports may contain the results of the summaries and data analysis performed in the Data Storage and Processing Architecture. User reports may be transmitted to the User interface and formatted in a suitable way for displaying them on the User interface. In this embodiment, User reports are displayed through the My Analytics module of the Mobile App 306, but other displays are suitable. User reports and data reported or collected from the user may also be transmitted to the Therapist Interface 320, if so agreed by the User. User reports transmitted to the Therapist Interface 320 are formatted for displaying on the Therapist Interface 320.

FIG. 3B shows an additional exemplary embodiment 301. In this embodiment, the User interface 304 may include a voice-based tool, the digital assistant 326, which may interact with the user. The digital assistant 326 may be accessed through the Mobile App 306 component of the User interface 304 and/or directly and/or through another User interface 304 component such as a Smart Speaker 328. Activation of the digital assistant 326 may be through a command, diagnostic measure or rating, therapeutic measure or rating, or action, or event, based on the interface used.

The digital assistant 326 may comprise a voice-based tool that helps patients manage their symptoms. When activated, the tool holds conversations with the user, and guides them through a series of grounding techniques. Patients can engage in conversations with the digital assistant 326 by using a Graphical User Interface (GUI), or a Voice User Interface (VUI). The Digital Assistant's 326 conversations utilize natural language processing (NLP) and machine learning (ML) to recognize the patient's emotions and to personalize its statements. These interactive and human like components make the digital assistant 326 an optimum tool for grounding. When the Digital Assistant 326 is in use, the system may track information about the patient's setting, life, symptoms, and overall mental health. This data collection helps enhance conversations and generates Patient Report Files for the user's therapist.

Upon activation, the digital assistant 326 may initiate a conversation with the User. Conversations can be over a Graphical User interface (GUI), a Voice User interface (VUI), or other interface allowing a conversation. The digital assistant's 326 conversations may utilize natural language processing (NLP) and machine learning (ML) to recognize the User's emotions and to personalize its responses. The digital assistant 326 may comprise artificial intelligence.

Users may use the digital assistant 326 component to help them manage and track symptoms like dysphoria, anxiety, depression, hypervigilance, avoidance, depersonalization, derealization, amnesia, and identity confusion.

FIG. 3C shows an additional exemplary embodiment 303. An additional exemplary embedment may comprise additional or alternative interfaces. For example, an additional exemplary embodiment may comprise a smart-speaker 328. The smart-speaker 328 may be configured to receive voice data or other data. Voice data may comprise journaling data or other data. For example, voice data may comprise a symptom rating. Further, voice data may comprise a command to open an application or a module of an application or perform an exercise such as a grounding exercise. Further, the smart speaker 328 may be configured to perform voice analysis or perform voice biometric analysis. In a further example, an additional exemplary embodiment may comprise a wearable device 330. The wearable device may comprise, for example, a smart watch. The wearable device 330 may be configured to receive voice data or other data. Voice data may comprise commands such as a command to open an application. Further, voice data may comprise journaling data. The wearable device 330 may be configured to receive or determine and transmit data such as voice data or biometric data. Biometric data may comprise a temperature or a pulse. The wearable device 330 may be in communication with the application 306. The wearable device 330 may be configured to transmit data to the application. For example, if the wearable device 330 detects a heartrate above a threshold, the wearable device may transmit that data to the application for logging or for other purposes such as launching the application.

The user interface 304 may be connected to other devices that interface or interact with the User, such as wearable devices 330, Smart Speakers 328, or voice enabled devices and the like but other devices may be used as well. A wearable device 330, smart speaker 328, voice-enabled device and the like may be a remote computing device, such as the electronic device 101 and/or the electronic device 201. These devices may complement or replace each other and/or the Mobile Device 302 connecting to the Mobile App 306. For example, when the user interface 304 is connected to a Smart Speaker 328, the Smart Speaker 328 may activate the Mobile App and/or the Digital Assistant 326 and/or provide data to the Data Storage and Processing Architecture 311. The Smart Speaker 328 may do this directly without activating other devices or with the support of other devices connected to the User interface.

Data sources may be expanded beyond the data provided from the User interface 304. These may include User data from other interactive sources and other third party sources. Examples are: wearable devices 330 connected or not to the User interface 304, electronic medical records, electronic health records, wellness apps, exercise data, insurance claims, etc. And analytical techniques may include several techniques such as: statistical analyses, computer algorithms, artificial intelligence, etc.

Personalized therapies generated by the Customized Therapy Generator 316 may include other therapies, in addition to VR Experiences for Exposure Therapy that can be personalized based on the data collected from the User interface. The VR Therapy Generator in previous embodiments is one example of the Customized Therapy Generator 316.

The database 310, electronic medical records, health record system and the like may be communicatively coupled. The coupling may be one-way, two-way, or any other suitable coupling arrangement. One-way connection may allow for the use of the EMR/EHR data 332 as input to the Database 310, already described. A two-way connection may allow for the writing or transfer of information collected through the overall system to the EMR/EHR 332. Such information may include reports 314 such as User reports, Therapist Reports, and other information available in the Database that is not present in the EMR/EHR system 332.

FIG. 3D shows an additional exemplary configuration and wireframes for the Mobile Application 306. The configuration 303 may comprise a navigable menu structure. The menu may comprise various components, for example: a my journal, a journal item, a my analytics, a my symptoms checklist, a settings and the like. Further, the configuration 303 may comprise a home. The home may comprise a grounding intermediary. The grounding intermediary may comprise a grounding tool #1, a grounding tool #2, a grounding tool #3, a grounding tool #4, and so on and the like. This exemplary embodiment should not be construed as limiting. Further, the home may comprise a symptom tracking intermediary. The symptom tracking intermediary may comprise a daily symptom tracking module, a trigger tracking module, a symptom checklist, a symptom checklist summary, a symptom selection module and the like. Further, the home may comprise a journaling intermediary. The journaling intermediary may comprise a journal reflections module, a custom entry module and the like. Further, the home may comprise a resources intermediary. The resources intermediary may comprise a glossary, a glossary item, hotlines and the like.

FIG. 3E shows an example navigational menu flow. As an example, the navigational menu flow may comprise a home and/or a menu. The home may comprise the home of FIG. 3D. The menu may comprise the menu of FIG. 3D. The home may comprise grounding exercises. The grounding exercises may comprise identity mindfulness tools. Further, the grounding exercises may comprise visuospatial tools. The grounding exercises may comprise somatic sensory tools. The home may further comprise symptom tracking which may comprise daily symptom tracking, symptom checklist and selection, and trigger tracking. Further, the home may comprise a journaling module. The journaling module may comprise a journal reflections module as well as a custom journal entry module. The home may further comprise resources. The resources may comprise a glossary and/or a hotlines. The menu may comprise a my journal which may comprise a record of grounding exercises, a record of journal entries, and/or a record of symptoms tracked. Further, the menu may comprise a my analytics comprising symptom ratings, crises and triggers module, crisis location module, symptom checklist average scores module and symptom checklist individual scores. The menu may further comprise settings. In this embodiment, the user can access a My Journal module, My Analytics module, or a Settings module from the Menu. Access to other modules from the Menu is also suitable.

The My Journal module may include at least but not limited to: a) a list of all automatically logged uses of grounding exercises on the Mobile App, b) a list of journal entries from the Journaling module, and c) the records from the Daily symptom Tracking module. Automatically logged entries from the use of grounding exercises on the Mobile App can be edited by the user by tapping on the record shown in My Journal. In this embodiment, the Journal Module has three components, but other configurations are suitable. My Journal module may comprise at least the following: a) a list of all automatically logged uses of grounding exercises on the Mobile App, b) a list of journal entries from the Journaling module, and c) the records from the Daily symptom Tracking module. Automatically logged entries from the use of grounding exercises on the Mobile App can be edited by the user by tapping on the record shown in My Journal. In this embodiment, the Journal Module has three components, but other configurations are suitable.

My Analytics module provides a summary presentation of the information collected through the Symptom Tracking and Journaling modules. The information has been summarized and may be further analyzed through the Data Storage and Processing Architecture (120) to provide insights.

The Settings module allows the user to change the settings for the Mobile App. In addition to the usual settings such as color, etc., as available, the Settings Module allows for a security PIN or password setup and change, and to enter and change information used for the proper functioning of the app, such as Name and Date of Birth. A PIN/password may be required to access the My Journal module, My Analytics module, and Settings module.

The My Analytics module provides a summary presentation of the information collected through the Symptom Tracking and Journaling modules. The information has been summarized and may be further analyzed through the Data Storage and Processing Architecture to provide insights.

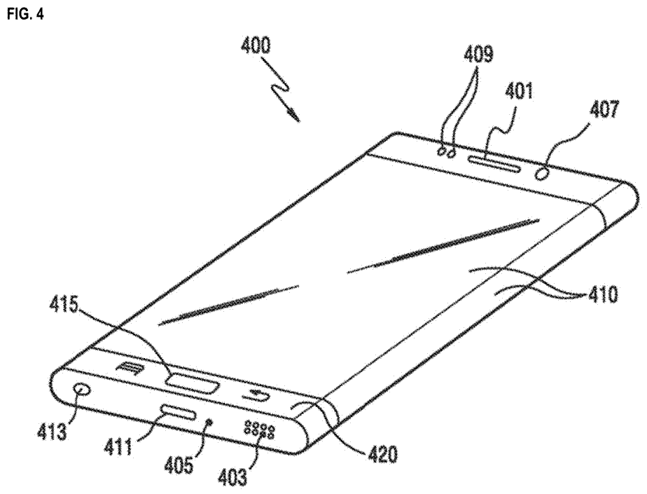

FIG. 4 illustrates an electronic device 400 according to various embodiments of the present disclosure. The electronic device 400 may be, for example, the electronic device 101 and/or the electronic device 201. According to various embodiments, the electronic device 400 may include a display 410, a housing (or a body) 420 to which the display 410 is coupled while the display 410 is seated therein, and an additional device formed on the housing 420 to perform the function of the electronic device 400. According to various embodiments, the additional device may include a first speaker 401, a second speaker 403, a microphone 405, sensors (for example, a front camera module 407 and an illumination sensor 409), communication interfaces (for example, a charging or data input/output port 411 and an audio input/output port 413), and a button 415. According to various embodiments, when the electronic device 400 and the AR/VR device 600 are connected through a wired communication scheme, the electronic device 400 may be connected based on at least some ports (for example, the data input/output port 411) of the communication interfaces.

According to various embodiments, the display 410 may include a flat display or a bended display (or a curved display) which can be folded or bent through a paper-thin or flexible substrate without damage. The bended display may be coupled to the housing 420 to remain in a bent form. According to various embodiments, the electronic device 400 may be implemented as a display device, which can be quite freely folded and unfolded such as a flexible display, including the bended display. According to various embodiments, in a Liquid Crystal Display (LCD), a Light Emitting Diode (LED) display, an Organic LED (OLED) display, or an Active Matrix OLED (AMOLED) display, the display 410 may replace a glass substrate surrounding liquid crystal with a plastic film to assign flexibility to be folded and unfolded.

According to various embodiments, the electronic device 400 may be connected to the AR/VR device 600. According to various embodiments, the electronic device 400 may be connected to other devices based on wireless communication (for example, Bluetooth or Bluetooth Low Energy (BLE)). The various devices may transmit and receive data such as device identifiers as well as diagnostic or therapeutic data and/or visual or audio data. Further, electronic device 400 may be configured to determine location or spatial or temporal data and may further be configured to transmit the same.

According to various embodiments, the electronic device 400 may generate relevant data for monitoring and/or diagnosis of user state (such as crisis state) and transmit the generated data to the AR/VR device 600.

According to various embodiments, the electronic device 400 may process an operation related to starting a measurement of (for example, acquire one or more indications from a user via a user interface) and displaying and/or transmitting a result. In an embodiment, the electronic device 400 can generate an instruction to cause the display 410 to display an exercise or an experience. In an embodiment, the instruction can cause the display to emit a stimulus according to a program stored on the electronic device 400. A user, observing the exercise or experience, may indicate via a touchscreen, button, and the like, of the electronic device 400 a reaction or other indication of a user state. For example, if an experience is too intense, a user may indicate thus. In another example, a user may indicate when he has complete an exercise. An exemplary exercise may be a color-finding exercise. The camera unit 291 may be configured to receive visual data. An instruction may appear on the display 410 which may instruct the user to identify an object having a certain color. The user may use the camera unit to identify the object having the certain color and may indicate as such.

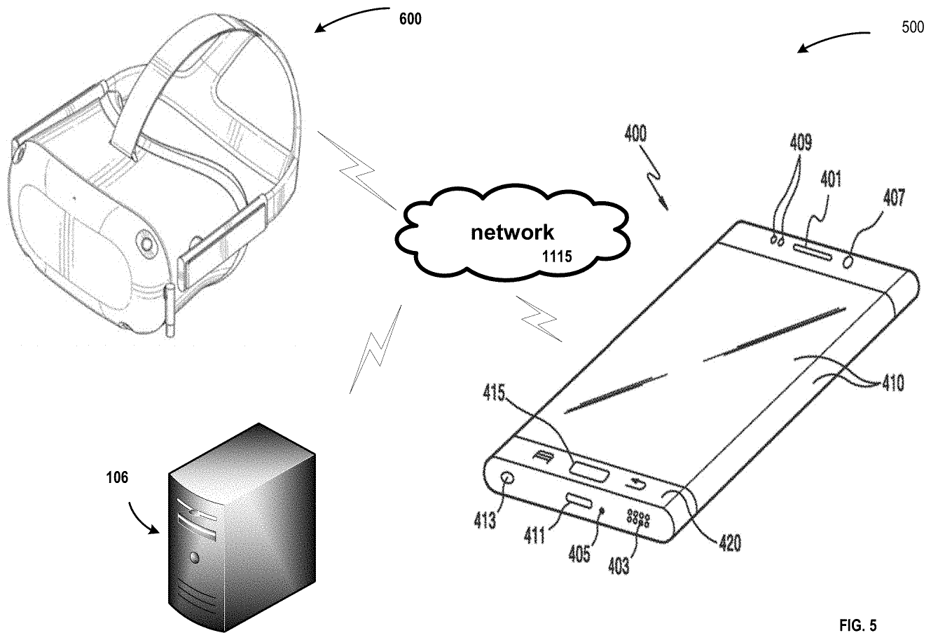

FIG. 5 illustrates an exemplary system 500 comprising the electronic device 400, the server 106, and an AR/VR device 600 according to various embodiments of the present disclosure. The operation and relevant screen examples of the electronic device 400 according to various embodiments will be described in detail with reference to the drawings below. The electronic device 400 and the AR/VR device may be communicatively coupled to a server 106 through a network (for example, network 1115). According to various embodiments, the electronic device 400 may include a display 410, a housing (or a body) 420 to which the display 410 is coupled while the display 410 is seated therein, and an additional device formed on the housing 420 to perform the function of the electronic device 400. According to various embodiments, the additional device may include a first speaker 401, a second speaker 403, a microphone 405, sensors (for example, a front camera module 407 and an illumination sensor 409), communication interfaces (for example, a charging or data input/output port 411 and an audio input/output port 413), and a button 415. According to various embodiments, when the electronic device 400 and the AR/VR device 600 are connected through a wired communication scheme, the electronic device 400 and the AR/VR device 600 may be connected based on at least some ports (for example, the data input/output port 411) of the communication interfaces.

According to various embodiments, the electronic device 400 may transmit at least data indicative of the PTSD symptoms, the indication(s), the time(s) and/or date(s), and the like, to the server 106 (e.g., a remote server). According to various embodiments, the server 106 may be connected to the electronic device 400 through wireless communication and may receive data from the electronic device 400 in real time. According to various embodiments, the AR/VR device 600 may display various UIs or GUIs based at least partially on the received data.

According to various embodiments, the electronic device 400 may include, for example, a smart phone and/or a tablet Personal Computer (PC). According to various embodiments, the electronic device 400 may display various User interfaces (UIs) or Graphical User interfaces (GUIs) related to using the AR/VR device 600.

According to various embodiments, the display 410 may include a flat display or a bended display (or a curved display) which can be folded or bent through a paper-thin or flexible substrate without damage. The bended display may be coupled to the housing 420 to remain in a bent form. According to various embodiments, the electronic device 400 may be implemented as a display device, which can be quite freely folded and unfolded such as a flexible display, including the bended display. According to various embodiments, in a Liquid Crystal Display (LCD), a Light Emitting Diode (LED) display, an Organic LED (OLED) display, or an Active Matrix OLED (AMOLED) display, the display 410 may replace a glass substrate surrounding liquid crystal with a plastic film to assign flexibility to be folded and unfolded.

According to various embodiments, the electronic device 400 may be connected to the AR/VR device 600. According to various embodiments, the electronic device 400 may be connected to the AR/VR device 600 based on wireless communication (for example, Bluetooth or Bluetooth Low Energy (BLE)).

According to various embodiments, the electronic device 400 may be connected to the AR/VR device 600, and may generate relevant data (for example, diagnostic or therapeutics of PTSD, including historical diagnostic or therapeutics) for monitoring and/or diagnosis of disease state and transmit the generated data to the AR/VR device 600 or the server 106.

In another embodiment, the methods described herein may be executed on a single device. For example, the methods described herein may be executed on the electronic device 400.

According to various embodiments, the electronic device 400 may process an operation related to starting a diagnostic or therapeutic function associated with a user condition such as PTSD using the other device 600 and displaying and/or transmitting a result to the server 106 or another device. In an embodiment, the electronic device 400 can send an instruction to the AR/VR device 600 to cause the AR/VR device 600 to output at least one stimulus (for example, a trigger or an experience). In an embodiment, the instruction can cause the AR/VR device 600 to output stimuli according to a pre-programmed function stored on the AR/VR device 600 or the server 106 or any other suitable device (for example, the database 310). A user, observing the stimulus emitted from the AR/VR device 600, may indicate via a touchscreen, button, and the like, of the electronic device 400. The stimulus and the user's reaction be logged by the electronic device 400. The instruction may indicate that the AR/VR device 600 is to repeat the function, or change functions. The diagnostic or therapeutic functions may be used to determine a state of the condition or disorder. The diagnostic or therapeutic functions may be added to a user profile as part of a historical record of PTSD diagnostic or therapeutics for a user. Further, inputs received may be added to the user profile. Additionally, inputs received may be used to update a database, for example database 310.

According to various embodiments, the electronic device 400 may receive AR/VR control information from the AR/VR device 600 and perform various operations (for example, configure one or more AR/VR experiences).

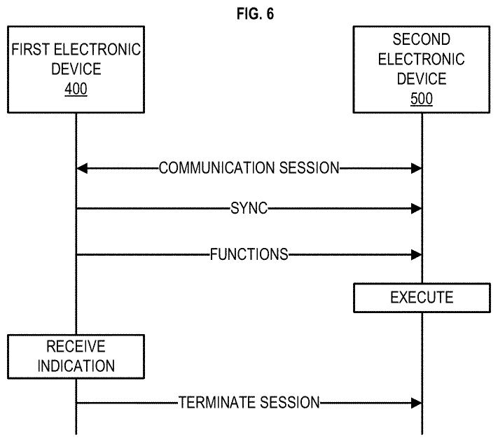

FIG. 6 illustrates a PTSD diagnostic or treatment process according to various embodiments of the present disclosure. The electronic device 400 (e.g., a smartphone) may open a communication session with a second electronic device such as AR/VR device 600 or server 106 (e.g., a remote server), or another electronic device such as 101, 102, or 104. Optionally, the electronic device 400 may send an instruction to the AR/VR device 600 to sync the internal clocks of both devices. The electronic device 400 may send an instruction to the AR/VR device 600 to cause the AR/VR device 600 or other suitable device such as device 101, 102, 104, or mobile phone 302 to initiate a PTSD diagnostic or therapeutic process or function. In various embodiments, the instruction may comprise one or more stimuli such as at least one trigger or experience. Further, the instruction may comprise at least one exercise. The AR/VR device 600, based on the instruction, may generate or send or output a stimulus, and may vary the stimulus according to the function. The electronic device 400 may receive an indication from a user. The indication may reflect an impression or the completion of a function and/or the like. The electronic device 400 may, based on the communication session, determine an action associated with the stimulus. In other embodiments, the electronic device 400 may query the AR/VR device for the parameters of the stimulus. The electronic device 400 may cause the PTSD diagnostic or therapeutic process to be repeated any number of times. Finally, the electronic device 400 may terminate the PTSD diagnostic or therapeutic process.

FIGS. 7A-7G illustrate exemplary user interfaces. Results from each iteration of the PTSD diagnostic or therapeutic process may be displayed to the user on the electronic device 400. The various user interfaces may facilitate a patient-system interaction or a therapist-system interaction (as seen in FIG. 7G). Therapists and/or other users may use a Therapy Administration Panel to conduct treatment sessions. This component connects with a mobile app or an advanced AR/VR headset, and allows the clinician to remotely start, stop, add, remove, or alter elements of the AR/VR Exposure Therapy Experience, without requiring the patient to remove his/her headset.

The therapist dashboard 322 may be embodied in an web application that gathers and visualizes data derived from the Digital Assistant 326. The web application may comprise mobile application 306. The web application may comprise separate logins for clinician-users. Users can access the application through an internet browser, and use the different features through a GUI. The dashboard allows the clinician to search through different patients and have the option to add a patient if one does not exist. Suggested therapy points generated for the clinician may be based upon information from the Patient Report Files. The dashboard also can help guide clinicians on their therapy sessions with their customer saving time. The dashboard may show graphs based upon Patient Report Files. The graphs on crisis location may be based upon the locations where users activated the Digital Assistant for the purpose of symptom management. The data may be presented on a map-like picture to show possible trends that may exist in the flashbacks or other crisis exhibited by the patient. The Crisis Assistant Report may indicate the type of experiences the patient is having and splitting it up by a number of different symptoms. This feature may summarize or analyze symptoms or events without having to count them manually based on a standardized journal. This can also help clinicians develop trend lines and easily see them visually without needing to do any further work themselves. Further, a Daily Average Symptom Ratings may be pulled from the Patient Report Files which requests metrics on a number of symptoms on a regular basis, for example, each day. The system may analyze this information for a clinician and provide up-to-date metrics on how a patient reports his or her symptoms. This can be used to develop trend lines and easily indicate a patient status over time. The right side of the dashboard includes notes and journals. This is a synopsis mimicking the qualitative information saved in Patient Report Files and allows clinicians to input voice, pictures, text comments and suggestions in the form of Therapist Entries. This feature also allows patients to see the daily rating and understand more in-depth journal usage from their patient. If a clinician would like to incorporate therapy session notes, they can also input the text for future reference. Furthermore, the Teletherapy Tab (not shown) may incorporate the clinician feature to setup and call their patient for a teletherapy session.

Described herein is a PTSD diagnostic process according to various embodiments of the present methods and systems. For example, a user may launch an application (e.g., software program) resident on the electronic device 400. The application (e.g., mobile application 306) may initiate a communication session with a server 106 or an AR/VR device 600. The user may engage a user interface 304 element on the electronic device 400 to calibrate the AR/VR device 600. In response, the AR/VR device 600 may output one or more stimuli or triggers or experiences or content or start a module. The user may engage a user interface 304 element on the electronic device 400 to start a PTSD diagnostic or therapeutic process or function. In response, the AR/VR device 600 may activate the displays to output a stimulus. The first or second electronic device may receive an input from a user.

FIG. 7H shows an exemplary interface comprising a welcome screen. The welcome screen may, for example, include options to perform grounding exercises, journaling, other resources or feedback.

FIG. 7I shows an exemplary interface comprising a home screen.

FIG. 7J shows an exemplary interface comprising my journal interface.

FIG. 7K shows an exemplary interface comprising an analytics interface.

FIG. 7L shows an exemplary interface comprising a further my analytics interface.

FIG. 7M shows an exemplary interface which may comprise, for example a name field, a personal phrase, a birthday, security, or location tracking.

FIG. 7N shows an exemplary interface which may comprise a listing of exercises including but not limited to flashback grounding, color finder, breathing exercises, vibration tool, bilateral stimulation, or 5, 4, 3, 2, 1 grounding.

FIG. 7O shows an exemplary interface which may comprise a welcome screen.

FIG. 7P shows an exemplary interface which may comprise a color-finding exercise.

FIG. 7Q shows an exemplary interface which may comprise a breathing exercise.

FIG. 7R shows an exemplary interface which may comprise a start function.

FIG. 7S shows an exemplary interface which may comprise a calming scene and may be accompanied by a calming audio output.

FIG. 7T shows an exemplary interface which may comprise a 5, 4, 3, 2, 1 grounding exercise.

FIG. 7U shows an exemplary interface which may comprise a journal prompt.

FIG. 7V shows an exemplary interface which may comprise a daily symptom tracking module.

FIG. 7W shows an exemplary interface which may comprise a custom journal entry.

FIG. 7X shows an exemplary interface which may comprise a journal reflections interface.

FIG. 7Y shows an exemplary interface which may comprise a trigger journal entry.

FIG. 7Z shows an exemplary interface which may comprise a PTSD checklist summary.

FIG. 7AA shows an exemplary interface which may comprise a resource selection interface.

FIG. 7BB shows an exemplary interface which may comprise a hotlines interface.

FIG. 7CC shows an exemplary interface which may comprise a glossary interface.

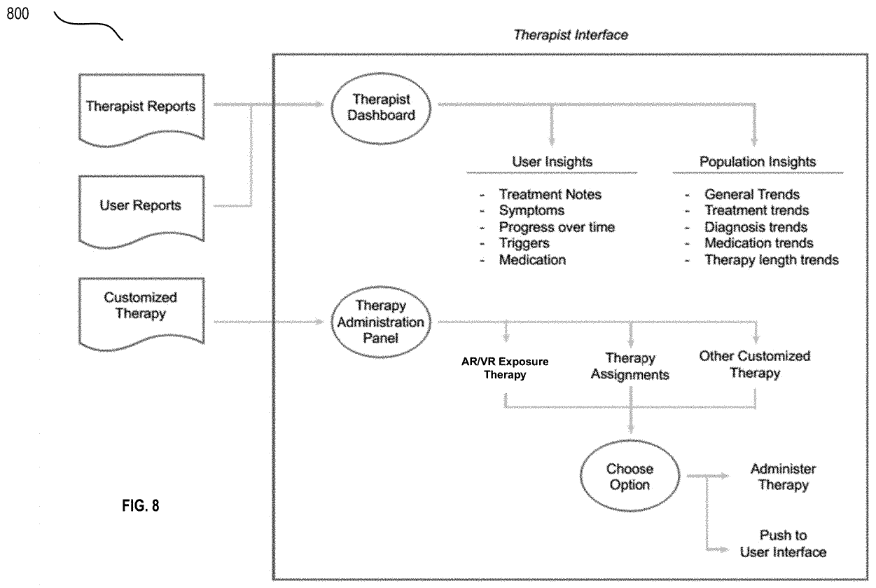

FIG. 8, shows a therapist interface process flow 800. The process flow 800 may be performed by the electronic device 101, the electronic device 201, the electronic device 400, or any other suitable device. The process flow 800 may comprise therapist reports, user reports, and customized therapy. The therapist reports and user reports may be in communication with a therapist dashboard. The therapist dashboard may display user insights such as treatment notes, symptoms, progress over time, triggers, medication and the like. Further, the therapist dashboard may comprise population insights. The population insights may comprise general trends, treatment trends, diagnosis trends, medication trends, therapy length trends, and the like. The process flow 800 may further comprise a therapy administration panel. The therapy administration panel may comprise AR/VR exposure therapy, therapy assignments, or other customized therapy. A user may select an option. The option may comprise for example, an administer therapy option or a push to user interface. The process flow 800 shows an exemplary flowchart for the Therapist Interface 320. The Therapist logs in and is authenticated. For each User that is also the Therapist's client and who has agreed to share his/her data with the Therapist, the Therapist can view the User's data and User reports, and use any VR Experiences generated for that User. The User's data and User reports are presented in a Therapist Dashboard 322. In addition, the Therapist Dashboard 322 displays the information from the Therapist Reports that include analysis across the Therapist's clients who are also Users. The Therapist Dashboard 322 also provides an area for the therapist's notes. Therapist's notes are stored in the database, for example database 310 but are not shared with the User. The VR Experiences are available through the Therapy Administration Panel. The Therapy Administration Panel connects with a mobile app or an advanced VR headset, and allows the therapist to remotely start, stop, add, remove, or alter elements of the VR Experience while in use.

A therapist may use the therapist dashboard 322 to better assess patients with mental health conditions such as PTSD, and plan better treatment sessions. Information stored in Patient Report Files and displayed on the therapist dashboard let therapists identify negative shifts in a patient's condition, track the effectiveness of new psychiatric drugs, and determine optimal treatment strategies.

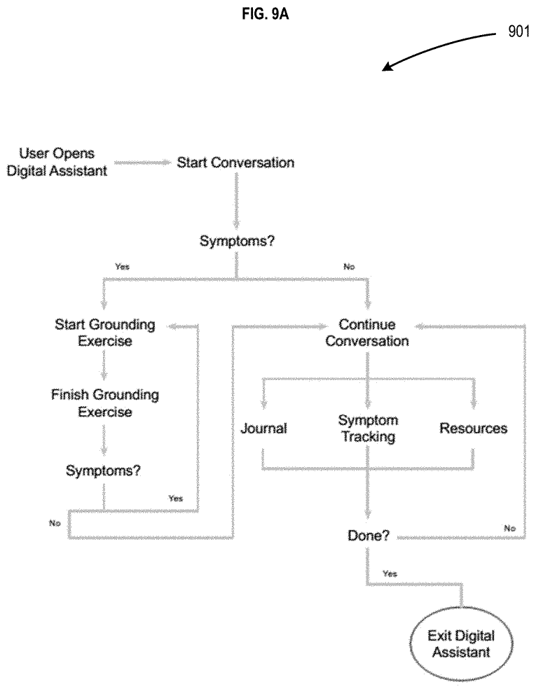

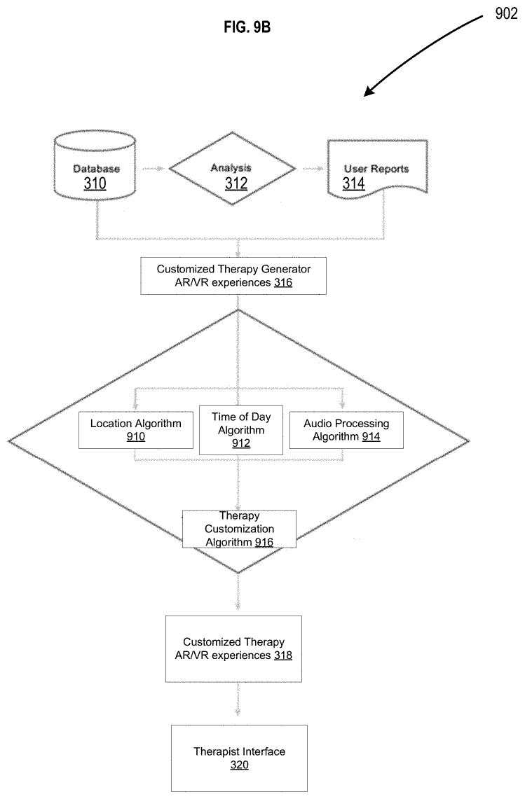

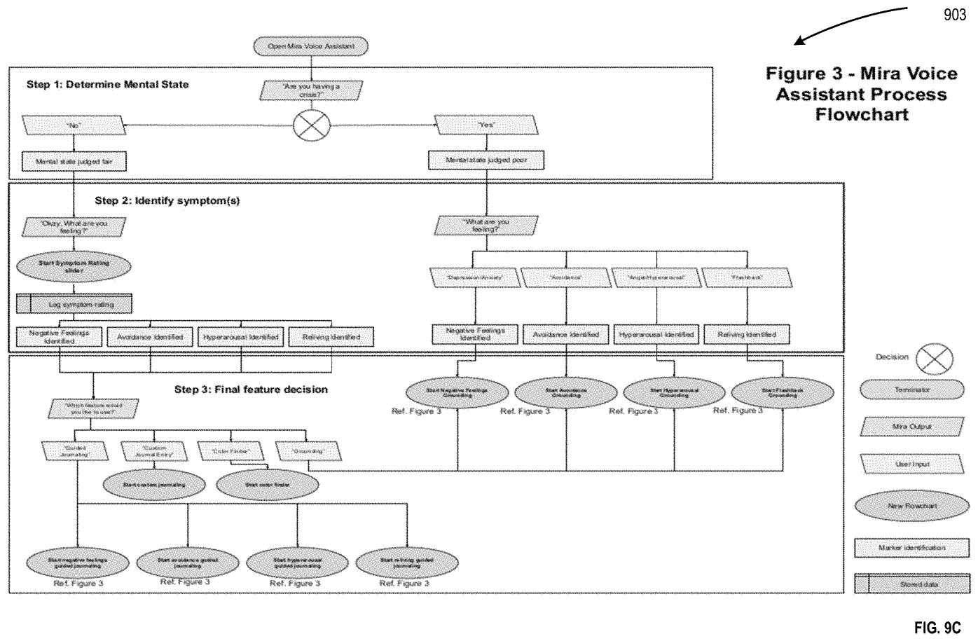

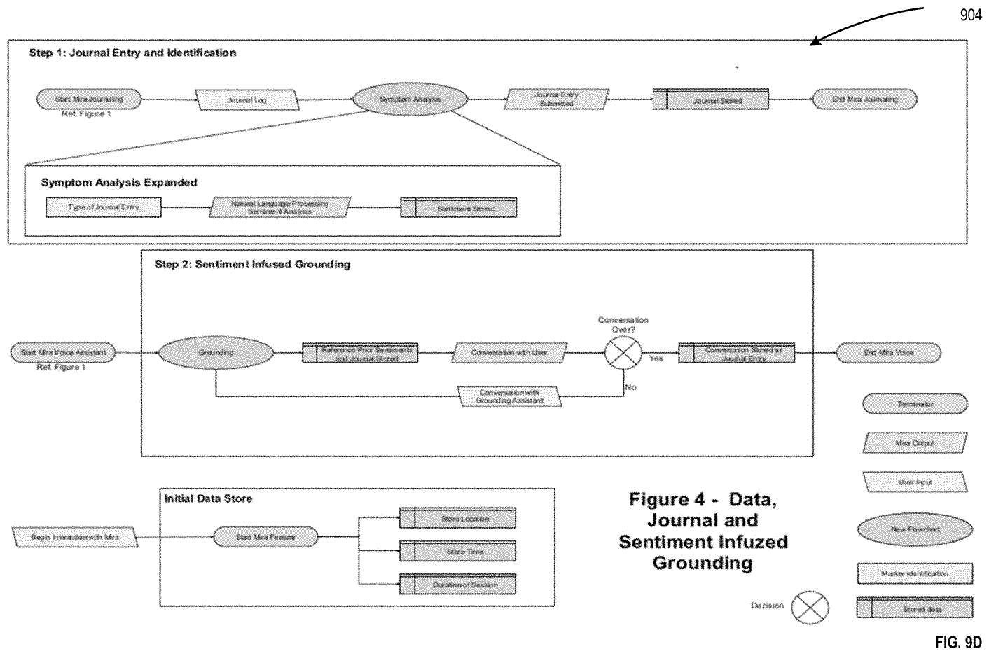

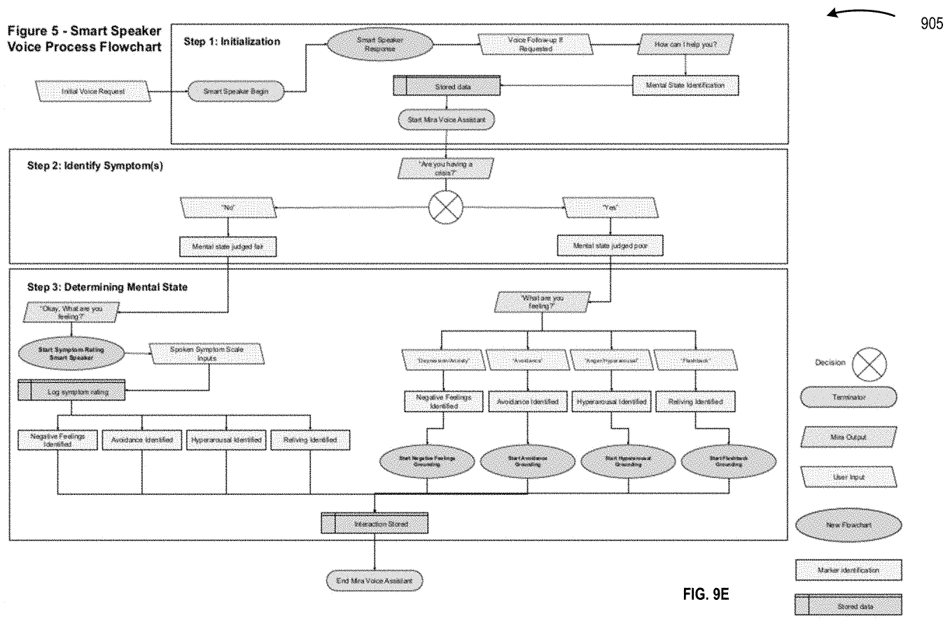

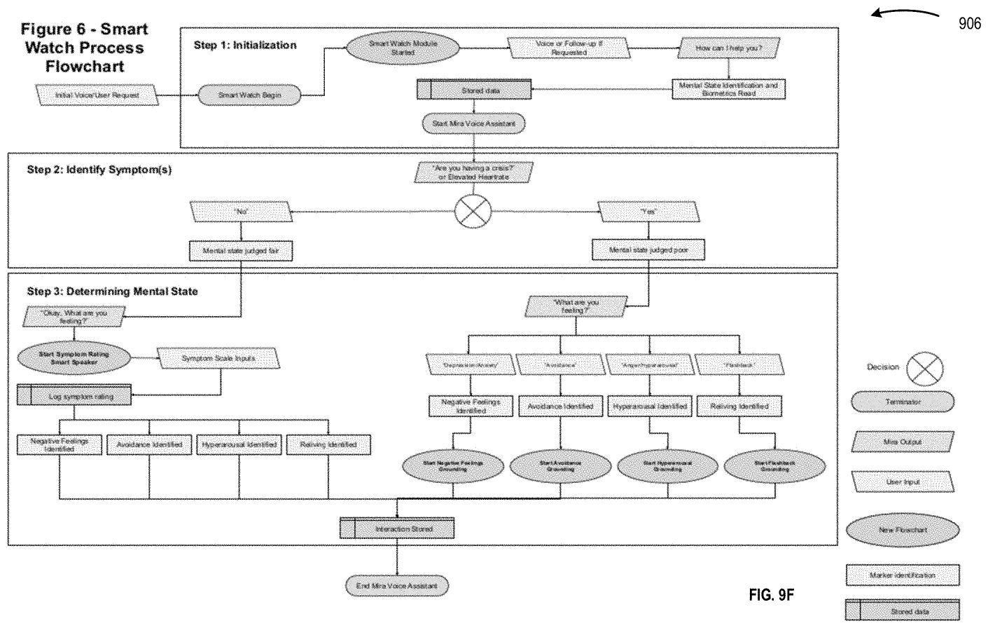

FIGS. 9A-9F show an example process flows. The example process flows shown in FIGS. 9A-9F may be performed using the electronic device 101, the electronic device 201, the electronic device 400, or any other suitable device. In process flow 901, a user may open a digital assistant, such as the digital assistant 326, and start a conversation. The conversation may comprise the user relaying symptoms. The conversation may be an audible exchange or it may comprise voice-to-text processing or it may be a digital conversation. Depending on the symptoms, the user may begin grounding exercises and after completing at least one grounding exercise, a determination may be made as to whether the symptoms are alleviated. Alternatively, based on the symptoms, the user may continue the conversation, may execute a journaling function, may execute a symptom tracking function, or may execute a resource function. Upon completion, the user may exit the digital assistant 326.

Process flow 901 shows a process flow for interactions with the digital assistant 326. The digital assistant 326 may first assess if the user is having symptoms. If the user if having or about to have a symptomatic event, the digital assistant 326 may guide the user to grounding exercises. Otherwise, the digital assistant 326 may guide the user to self-assessment, journaling, or other features and resources of the system, based on the conversation with the user.

In process flow 901, conversations with the digital assistant may be logged and the content of the conversations may be used to inform future conversations with the Digital Assistant 326 and as input to the Database 310 in the Data Storage and Processing Architecture 311. Conversations with the digital assistant 326 may be logged automatically or manually. They may be stored in a database, such as the database 310.

The digital assistant 326 adds interactivity to the user interface and provides a more personalized experience that is closer to human interactions. Therefore, the digital assistant 326 provides another option for self-management of symptoms that may be more effective or preferred by some users. The digital assistant 326, through the conversations with the user, also collects richer data on the user status, well-being, symptoms, etc. than other embodiments. Thus, user reports and therapist reports 314 can provide further insights.

FIG. 9B shows an example process flow 902. In process flow 902, a database 310 may be in communication with an analysis engine, for example analysis engine 312, which may generate reports 314 such as user reports or therapist reports. A customized therapy generator 316, for example customized therapy generator 316 may comprise a location algorithm 910, a time of day algorithm 912, and an audio processing algorithm 914. The location algorithm 910 may receive location data, for example, from a user device, and may retrieve or receive or generate or output location data and/or spatial data and the like. Location data may be transmitted. Likewise, the time of day algorithm may receive temporal data as inputs and retrieve or receive or generate or output temporal data. Temporal data may be transmitted. In a similar fashion, the audio processing algorithm may receive audio data as inputs and retrieve or receive or generate or output audio data as an output. Audio data may be transmitted. The aforementioned algorithms may be in communication so as to develop and execute a therapy customization algorithm 916. The therapy customization algorithm may be used to generate a customized therapy 318 (for example an AR experience or a VR experience). The customized therapy experience 318 may be in communication with a therapist interface 320 so as to execute a diagnostic or therapeutic function. The process flow 902 describes the flowchart for the AR/VR Experience Generator to create personalized AR/VR Experiences based on the User reports and User's data. Information captured through the User interface on triggers, time and location of symptoms, and comments or recordings on the symptomatic event may be used to create AR/VR Experiences that mimic the environment that triggered the symptomatic event. These experiences may show the real-world location of the symptomatic event, depict different times of day through lighting changes, and incorporate background noises related to the real world location. The created AR/VR Experience is sent to the Therapist Interface of the therapist associated with the User. The system may then push the experience to the user interface.

Further, a therapist may use the system to execute the process flow 902. As an example, a therapist may administer personalized AR/VR Exposure Therapy to a patient. At the start of the session, the clinician securely logs in, and then selects the patient that he/she would like to administer treatment to. After selecting a patient, the clinician is taken to a Therapy Administration Panel. This panel displays data stored in the AR/VR Therapy Database associated with the previously selected patient. From this database, a clinician can select pre-compiled AR/VR Exposure Therapy Experiences, or select individual elements to customize a new AR/VR Exposure Therapy Experience. Once a desired AR/VR Exposure Therapy Experience is selected, the clinician can use the Therapy Administration Panel to start the treatment session. The designated AR/VR Exposure Therapy Experience is then shared to an advanced AR/VR headset or to a patient's mobile application that is placed into a basic AR/VR headset. The patient must wear one of those AR/VR headsets while the clinician conducts the therapy session. During treatment, clinicians can use the Therapy Administration Panel to remotely pause the video, alter individual elements, move the patient's first person perspective, or end the session.

The system may generate and output personalized treatment experiences. Unlike other VR therapy options, the system provides personalized experiences based on stimuli and inputs that may trigger a user in his or her daily life. Further, the delivery of the AR/VR experience is in a controllable, confined virtual space. Participating in Exposure Therapy helps patients develop normal relationships with the everyday locations and stimuli that would ordinarily escalate their symptoms.

FIG. 9C shows an example process flow 903 which may comprise determining a mental state, identifying symptoms, and final feature decision. The process flow 903 may be readily understood by one skilled in the art with reference to FIG. 9C.

FIG. 9D shows an example process flow 904 which may comprise journal entry and identification, symptom analysis, sentiment infused grounding, and data storage. Process flow 904 may be readily understood by one skilled in the art with reference to FIG. 9D.

FIG. 9E shows an example process flow 905 which may comprise initialization, symptom identification and determining a mental state. The process flow 905 may incorporate a smart speaker, for example smart speaker 328. The process flow 905 may be readily understood by one skilled in the art with reference to FIG. 9E. In this embodiment, Users can activate the digital assistant 326 by saying a “wake word” such as “Hey, Google,” “Siri,” “Alexa,” or “Mira,” and navigate different features through a voice user interface. The voice is differentiated from the mobile app because all interactions will be spoken to the system by users voice. The Digital Assistant 326 will activate and run a similar pattern as found in FIGS. 3 and 4 to help ground the user. Prompts also incorporate previous stored interactions in prior journal entries to help ground the user. The remaining of the prompts will further go through the full feature set of the Digital Assistant and engage the user in grounding or other features integrated into the smart speaker embodiment.

FIG. 9F shows an example process flow 906 which may comprise initialization, symptom identification, and determining a mental state. The process flow 906 may incorporate a wearable device 330 as a smart watch. Users can activate features of the digital assistant 326 through a GUI, programmed shortcut gestures, or voice. The smartwatch module will begin upon activation and then prompt for follow-up from the user. This particular embodiment can also identify dissociation and other negative changes in the user's mental status by incorporating biometrics. These biometrics track heart rate, skin conductors, and other stress indicators. In this embodiment, when the digital assistant identifies that a patient is entering a state of crisis, it can ask the user if he/she would like to activate particular grounding tools. If the user agrees, the digital assistant would then guide the individual through the prompts and grounding techniques found in FIG. 3. The Digital Assistant 326 will deactivate once the user has completed the session and interaction has been stored. The process flow 906 may be readily understood by one skilled in the art with reference to FIG. 9F.

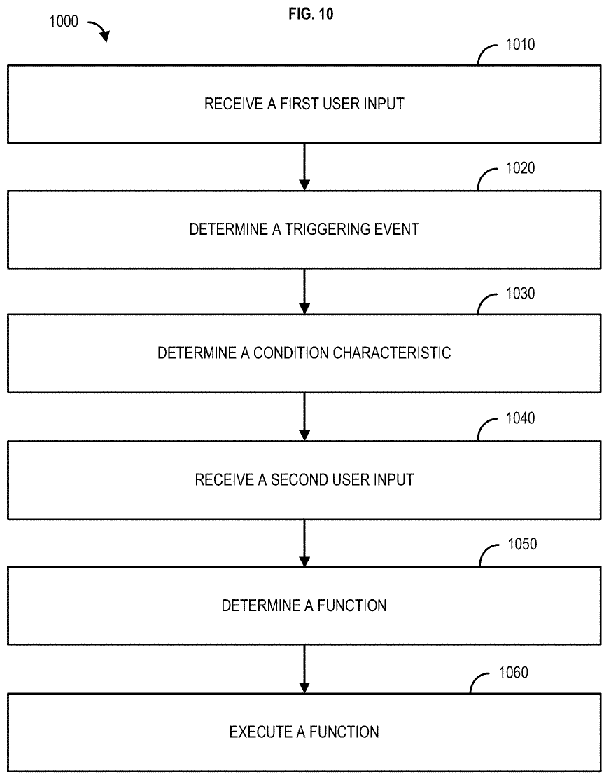

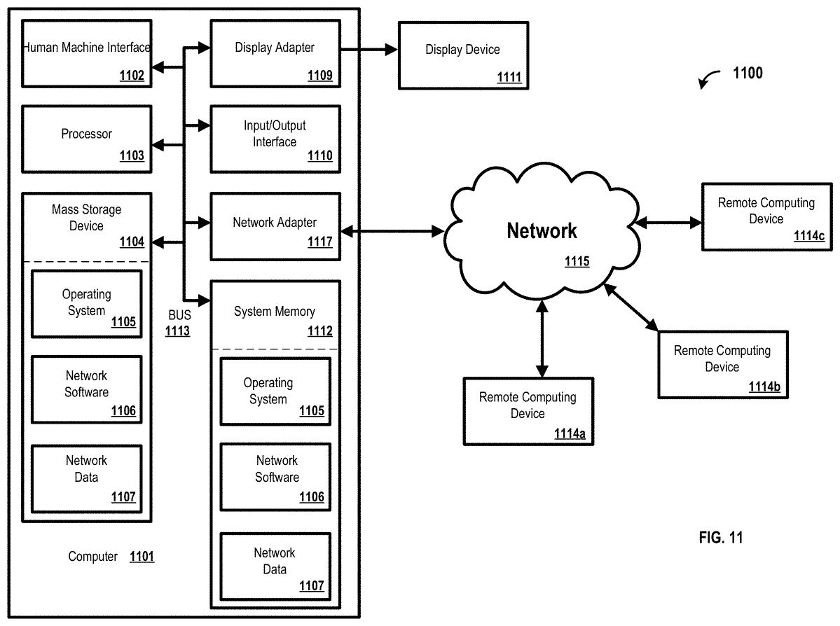

FIG. 10 shows an example method 1000. The method 1000 may be performed using the electronic device 101, the electronic device 201, the electronic device 400, or any other suitable device. The method 1000 may comprise receiving a first user input at 1010. Receiving a first user input may comprise receiving an indication comprising an identifier. The identifier may be associated with a particular user. Further, the identifier may comprise a symptom or the like. The method 1000 may comprise determining a triggering event at 1020. Determining a triggering event may comprise determining a symptom or a degree of a symptom or an event or a stimuli. The method 1000 may comprise determining condition characteristics at 1030. Determining a condition characteristic may comprise selecting from a list of symptoms or characteristics associated with a disorder such as PTSD. Further, determining a condition characteristic may comprise receiving an input such as a journal entry. The method 1000 may comprise receiving an additional user input at 1040. Receiving an additional user input may comprise receiving a diagnostic or therapeutic selection. For example, a user may select an option for diagnoses whereby the user may enter information. Additionally or alternatively, the user may select a therapeutic option which may comprise presenting various therapeutic functions to be executed. The method 1000 may comprise determining, at least one of a plurality of functions at 1050. The method 1000 may comprise executing at least one function at 1060. Executing a function may comprise receiving or transmitting diagnostic information such as a symptom, a user identifier, a device identifier, a time of day and the like. Additionally or alternatively, executing a function may comprise executing a therapeutic function such as executing a color-finding function or a breathing exercise function or a haptic function or the like.

While the methods and systems have been described in connection with specific examples, it is not intended that the scope be limited to the particular embodiments set forth, as the embodiments herein are intended in all respects to be illustrative rather than restrictive. Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice described herein. It is intended that the specification and examples be considered as exemplary only, with a true scope and spirit being indicated by the following claims.

What is claimed is:

1. A method comprising:
determining, based on a first user input, a triggering event associated with a condition;
determining, based on the condition, condition characteristics;
determining, based on the condition characteristics, at least one of a plurality of options associated with one or more of a plurality of diagnostic functions or a plurality of therapeutic functions;
receiving, based on the at least one of the plurality of options, an additional user input associated with the one or more of the plurality of diagnostic functions or the plurality of therapeutic functions;
determining, based on the additional user input, at least one of the plurality of diagnostic functions or at least one of the plurality of therapeutic functions; and
executing the at least one diagnostic function or the at least one therapeutic functions.

2. The method of claim 1, wherein the plurality of therapeutic functions comprise one or more of AR/VR experiences or grounding exercises.

3. The method of claim 1, further comprising generating, based on the condition characteristics, treatment decision support wherein the treatment decision support comprises a custom therapy recommendation.

4. The method of claim 1, further comprising sending, based on the at least one diagnostic function or the at least one therapeutic function, data indicative of the at least one diagnostic function or the at least one therapeutic function.

5. The method of claim 1, wherein the plurality of diagnostic functions comprise functions to identify a mental condition based on receiving diagnostic data.

6. The method of claim 1, wherein executing the at least one therapeutic function comprises generating a user-specific experience based on diagnostic data.

7. The method of claim 1, wherein the executing the at least one diagnostic function or the at least one therapeutic function comprises determining at least one of audio or visual data based on a user input associated with at least one of spatial or temporal information.

8. A system comprising:
a user device configured to:
receive a first user input;
determine, based on the first user input; a triggering associated with a condition;
receive, based on the at least one of the plurality of options, an additional user input associated with the at least one of the plurality of diagnostic functions or the plurality of therapeutic functions;
execute, the at least one diagnostic function or the at least one therapeutic functions;
the computing device configured to:
determine, based on the condition, condition characteristics;
determine, based on the condition characteristics, at least one of a plurality of options associated with one or more of a plurality of diagnostic functions or a plurality of therapeutic functions;
determine based on the additional user input, at least one of the plurality of diagnostic functions or at least one of the plurality of therapeutic functions.

9. The system of claim 7, wherein the plurality of therapeutic functions comprise one or more of AR/VR experiences or grounding exercises.

10. The system of claim 7, wherein the computing device is further configured to generate, based on the condition characteristics, treatment decision support wherein the treatment decision support comprises a custom therapy recommendation.

11. The system of claim 7, is further configured to send, based on the at least one diagnostic function or the at least one therapeutic function, data indicative of the at least one diagnostic function or the at least one therapeutic function.

12. The system of claim 7, wherein the plurality of diagnostic functions comprise functions to identify a mental condition based on receiving diagnostic data.

13. The system of claim 7, wherein to execute the at least one diagnostic function or at least one therapeutic function, the computing device is further configured to generate a user-specific experience based on diagnostic data.

14. The system of claim 7, wherein to execute the at least one diagnostic function or the at least one therapeutic function, the computing device is further configured to determining at least one of audio or visual data based on a user input associated with at least one of spatial or temporal information.

15. An apparatus comprising:
one or more processors; and
memory storing processor executable instructions that, when executed by the one or more processors, cause the apparatus to:
receiving a first user input;
determining, based on the first user input; a triggering event associated with a condition;
determining, based on the condition, condition characteristics;
determining, based on the condition characteristics, at least one of a plurality of options associated with one or more of a plurality of diagnostic functions or a plurality of therapeutic functions;
receiving, based on the at least one of the plurality of options, a second user input associated with one or more of the plurality of diagnostic functions or the plurality of therapeutic functions;
determining based on the second user input, at least one of the plurality of diagnostic functions or at least one of the plurality of therapeutic functions; and
executing the at least one diagnostic function or the at least one therapeutic functions.

16. The apparatus of claim 15, wherein the plurality of therapeutic functions comprise at least one or more of AR/VR experiences.

17. The apparatus of claim 15, wherein the processor executable instructions when executed by the one or more processors, further cause the apparatus to generate, based on the condition characteristics, treatment decision support wherein the treatment decision support comprises a custom therapy recommendation.

18. The apparatus of claim 15, wherein the processor executable instructions that, when executed by the one or more processors, cause the apparatus to execute the at least one diagnostic function or the least one therapeutic function, cause the apparatus to execute the at least one function by determining at least one of audio or visual data based on a user input associated with at least one of spatial or temporal information.

19. The apparatus of claim 15, wherein the processor executable instructions that, when executed by the one or more processors, cause the apparatus to execute the at least one diagnostic function or the least one therapeutic function, cause the apparatus to execute the at least one function by determining diagnostic data.

20. The apparatus of claim 15, wherein the processor executable instructions when executed by the one or more processors, further cause the apparatus to send, based on the at least one diagnostic function of therapeutic function, data indicative of the at least one diagnostic function or therapeutic function.
